SEQUENCE LISTING (62 sequences)

SEQ ID NO: 1 - DNA, 21 bp, Homo sapiens
  CDS (1)..(21)
tacaatgagcccaaggtgacg

SEQ ID NO: 2 - protein, 7 aa, Homo sapiens
YNEPKVT

SEQ ID NO: 3 - DNA, 21 bp, Homo sapiens
  CDS (1)..(21)
agcctcccgcaacgcctggct

SEQ ID NO: 4 - protein, 7 aa, Homo sapiens
SLPQRLA

SEQ ID NO: 5 - DNA, 21 bp, Homo sapiens
  CDS (1)..(21)
gctggccgtgacctcaaacgc

SEQ ID NO: 6 - protein, 7 aa, Homo sapiens
AGRDLKR

SEQ ID NO: 7 - DNA, 21 bp, Rattus norvegicus
  CDS (1)..(21)
tacaatgagcccaaggtcaca

SEQ ID NO: 8 - protein, 7 aa, Rattus norvegicus
YNEPKVT

SEQ ID NO: 9 - DNA, 21 bp, Rattus norvegicus
  CDS (1)..(21)
aacctaccccaacgcctggca

SEQ ID NO: 10 - protein, 7 aa, Rattus norvegicus
NLPQRLA

SEQ ID NO: 11 - DNA, 21 bp, Rattus norvegicus
  CDS (1)..(21)
gcaggccgtgatctgaagcgc

SEQ ID NO: 12 - protein, 7 aa, Rattus norvegicus
AGRDLKR

SEQ ID NO: 13 - DNA, 24 bp, Homo sapiens
  CDS (1)..(24)
ctgcagaagttccgcggctcctcc

SEQ ID NO: 14 - protein, 8 aa, Homo sapiens
LQKFRGSS

SEQ ID NO: 15 - DNA, 24 bp, Rattus norvegicus
  CDS (1)..(24)
ctgcagaagttccgaggttcctca

SEQ ID NO: 16 - protein, 8 aa, Rattus norvegicus
LQKFRGSS

SEQ ID NO: 17 - DNA, 15 bp, Homo sapiens
  CDS (1)..(15)
aagttccgcggctcc

SEQ ID NO: 18 - protein, 5 aa, Homo sapiens
KFRGS

SEQ ID NO: 19 - DNA, 15 bp, Rattus norvegicus
  CDS (1)..(15)
aagttccgaggttcc

SEQ ID NO: 20 - protein, 5 aa, Rattus norvegicus
KFRGS

SEQ ID NO: 21 - DNA, 15 bp, Homo sapiens
  CDS (1)..(15)
cagaagttccgcggc

SEQ ID NO: 22 - protein, 5 aa, Homo sapiens
QKFRG

SEQ ID NO: 23 - DNA, 15 bp, Rattus norvegicus
  CDS (1)..(15)
cagaagttccgaggt

SEQ ID NO: 24 - protein, 5 aa, Rattus norvegicus
QKFRG

SEQ ID NO: 25 - DNA, 12 bp, Homo Sapiens
  CDS (1)..(12)
aagttccgcggc

SEQ ID NO: 26 - protein, 4 aa, Homo Sapiens
KFRG

SEQ ID NO: 27 - protein, 9 aa, Artificial
  Cysteines added to both termini of a protein with the amino acid sequence of SEQ ID NOs2 or 8 for cyclization
  MOD_RES (1)..(1): ACETYLATION
  MOD_RES (9)..(9): AMIDATION
CYNEPKVTC

SEQ ID NO: 28 - protein, 9 aa, Artificial
  Cysteines added to both termini of a protein with the amino acid sequence of SEQ ID NO4 for cyclization
  MOD_RES (1)..(1): ACETYLATION
  MOD_RES (9)..(9): AMIDATION
CSLPQRLAC

SEQ ID NO: 29 - protein, 9 aa, Artificial
  Cysteines added to both termini of a protein with the amino acid sequence of SEQ ID NO10 for cyclization
  MOD_RES (1)..(1): ACETYLATION
  MOD_RES (9)..(9): AMIDATION
CNLPQRLAC

SEQ ID NO: 30 - protein, 9 aa, Artificial
  Cysteines added to both termini of a protein with the amino acid sequence of SEQ ID NOs6 or 12 for cyclization
  MOD_RES (1)..(1): ACETYLATION
  MOD_RES (9)..(9): AMIDATION
CAGRDLKRC

SEQ ID NO: 31 - protein, 10 aa, Artificial
  Cysteines added to both termini of a protein with the amino acid sequence of SEQ ID NOs14 or 16 for cyclization
  MOD_RES (1)..(1): ACETYLATION
  MOD_RES (10)..(10): AMIDATION
CLQKFRGSSC

SEQ ID NO: 32 - protein, 7 aa, Artificial
  Cysteines added to both termini of a protein with the amino acid sequence of SEQ ID NOs18 or 20 for cyclization
  MOD_RES (1)..(1): ACETYLATION
  MOD_RES (7)..(7): AMIDATION
CKFRGSC

SEQ ID NO: 33 - protein, 7 aa, Artificial
  Cysteines added to both termini of a protein with the amino acid sequence of SEQ ID NOs22 or 24 for cyclization
  MOD_RES (1)..(1): ACETYLATION
  MOD_RES (7)..(7): AMIDATION
CQKFRGC

SEQ ID NO: 34 - protein, 6 aa, Artificial
  Cysteines added to both termini of a protein with the amino acid sequence of SEQ ID NO26 for cyclization
  MOD_RES (1)..(1): ACETYLATION
  MOD_RES (6)..(6): AMIDATION
CKFRGC

SEQ ID NO: 35 - protein, 8 aa, Artificial
  Peptide from nerve growth factor, with cysteines added to both termini for cyclization
  MOD_RES (1)..(1): ACETYLATION
  MOD_RES (8)..(8): AMIDATION
CTDKGKEC

SEQ ID NO: 36 - protein, 13 aa, Homo sapiens
LWAWLQKFRGSSS

SEQ ID NO: 37 - protein, 6 aa, Artificial
  Reverse sequence (retro-inverso) of shortened version of, e.g., SEQ ID NOs14 or 31, comprising D-amino acids in positions 1, 3, 4, 5, and 6 (s, r, f, k, and q, respectively), and cyclized by homodetic cyclization; can be represented as c[sGrfkq]
  MISC_FEATURE (1)..(1): D-Ser
  MISC_FEATURE (3)..(3): D-Arg
  MISC_FEATURE (4)..(4): D-Phe
  MISC_FEATURE (5)..(5): D-Lys
  MISC_FEATURE (6)..(6): D-Gln
XGXXXX

SEQ ID NO: 38 - DNA, 40 bp, Homo sapiens
ctgggcctggctgcaggacttcgatggctcctcctccgag

SEQ ID NO: 39 - DNA, 40 bp, Homo sapiens
ctcggaggaggagccatcgaagtcctgcagccaggcccag

SEQ ID NO: 40 - DNA, 51 bp, Homo sapiens
ctctgggcctggctgcaggcgttcgccggctcctcctccgaggtgccctgc

SEQ ID NO: 41 - DNA, 51 bp, Homo sapiens
gcagggcacctcggaggaggagccggcgaacgcctgcagccaggcccagag

SEQ ID NO: 42 - DNA, 40 bp, Homo sapiens
ctctgggcctggctgcaggcgttccgcggctcctcctccg

SEQ ID NO: 43 - DNA, 40 bp, Homo sapiens
cggaggaggagccgcggaacgcctgcagccaggcccagag

SEQ ID NO: 44 - DNA, 40 bp, Homo sapiens
gcctggctgcagaagttcgccggctcctcctccgaggtgc

SEQ ID NO: 45 - DNA, 40 bp, Homo sapiens
gcacctcggaggaggagccggcgaacttctgcagccaggc

SEQ ID NO: 46 - protein, 7 aa, Artificial
  Cysteines added to both termini of the protein for cyclization
  MOD_RES (1)..(1): ACETYLATION
  MOD_RES (7)..(7): AMIDATION
CQAFRGC

SEQ ID NO: 47 - protein, 7 aa, Artificial
  Cysteines added to both termini of the protein for cyclization
  MOD_RES (1)..(1): ACETYLATION
  MOD_RES (7)..(7): AMIDATION
CQKARGC

SEQ ID NO: 48 - protein, 7 aa, Artificial
  Cysteines added to both termini of the protein for cyclization
  MOD_RES (1)..(1): ACETYLATION
  MOD_RES (7)..(7): AMIDATION
CQKFAGC

SEQ ID NO: 49 - protein, 7 aa, Artificial
  Cysteines added to both termini of the protein for cyclization
  MOD_RES (1)..(1): ACETYLATION
  MOD_RES (7)..(7): AMIDATION
CQKFRAC

SEQ ID NO: 50 - DNA, 43 bp, Homo sapiens
ctgctcttggtcatgtgaacgtaacgataaaggagctcaggcg

SEQ ID NO: 51 - DNA, 43 bp, Homo sapiens
cgcctgagctcctttatcgttacgttcacatgaccaagagcag

SEQ ID NO: 52 - protein, 8 aa, Homo sapiens
FQRARVSS

SEQ ID NO: 53 - DNA, 39 bp, Homo sapiens
ctgggcccggggctgttcgagggcctggctgccctgcag

SEQ ID NO: 54 - DNA, 39 bp, Homo sapiens
ctgcagggcagccaggccctcgaacagccccgggcccag

SEQ ID NO: 55 - DNA, 39 bp, Homo sapiens
gcctggctgcagaagttcgagggctcctcctccgaggtg

SEQ ID NO: 56 - DNA, 39 bp, Homo sapiens
cacctcggaggaggagccctcgaacttctgcagccaggc

SEQ ID NO: 57 - DNA, 39 bp, Homo sapiens
gtgcccgagcgcgccttcgaggggctgcacagcctcgac

SEQ ID NO: 58 - DNA, 39 bp, Homo sapiens
gtcgaggctgtgcagcccctcgaaggcgcgctcgggcac

SEQ ID NO: 59 - DNA, 1628 bp, Artificial
  Fusion of nucleotides encoding the first 310 amino acids of NgR1 with nucleotides encoding human Fc region
gtcgacgccaccatgaaattcttagtcaacgttgcccttgtttttatggtcgtgtacatttcttacatctatgcctgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgcggccgctggagacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgaattc

SEQ ID NO: 60 - DNA, 6964 bp, Artificial
  Expression vector comprising fusion of nucleotides encoding the first 310 amino acids of human NgR1 and a human fc portion
tactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccaagctctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgacgccaccatgaaattcttagtcaacgttgcccttgtttttatggtcgtgtacatttcttacatctatgcctgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgcggccgctggagacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgaattctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattgctcgagccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcattttttataagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccggccaacggtctggtgacccggctgcgagagctcggtgtacctgagacgcgagtaagcccttgagtcaaagacgtagtcgttgcaagtccgcaccaggtactgatcatcgatgctagaccgtgcaaaaggagagcctgtaagcgggcactcttccgtggtctggtggataaattcgcaagggtatcatggcggacgaccggggttcgaaccccggatccggccgtccgccgtgatccatccggttaccgcccgcgtgtcgaacccaggtgtgcgacgtcagacaacgggggagcgctccttttggcttccttccaggcgcggcggctgctgcgctagcttttttggcgagctcgaattaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttgtatac

SEQ ID NO: 61 - DNA, 334 bp, Homo sapiens
  misc_feature (176)..(176): n is a, c, g, or t
taagaaaagagctctttattccacgtcgtccgatatttttacacaagtaaaataaaatgcatatctctatataccgcgatctgggtgggaggcggcgttctggaacaaacgctgccgccgaaccctgtaaacatgatggggtgggaaaatgggggtcggcggcaggcgtccatcanggaaggacctggcctggcctgccccacgggtcggccgcccggctggcttggcggcgtggagagagaccccgtatgtacacacacctggctgctgagcacgctcttgtgtccgcttggggtcagcagggcccaagcactgtccacagcaccagcgccag

SEQ ID NO: 62 - DNA, 1924 bp, Homo sapiens
ctgtgcgccctgcgcgccctgcgcacccgcggcccgagcccagccagagccgggcggagcggagcgcgccgagcctcgtcccgcggccgggccggggccgggccgtagcggcggcgcctggatgcggacccggccgcggggagacgggcgcccgccccgaaacgactttcagtccccgacgcgccccgcccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccagcggacacaagagcgtgctcagcagccaggtgtgtgtacatacggggtctctctccacgccgccaagccagccgggcggccgacccgtggggcaggccaggccaggtcctccctgatggacgcctgccgcccgccacccccatctccaccccatcatgtttacagggttcggcggcagcgtttgttccagaacgccgcctcccacccagatcgcggtatatagagatatgcattttattttacttgtgtaaaaatatcggacgacgtggaataaagagctcttttcttaaaaaaa

(19) United States
(12) Patent Application Publication
(10) Pub. No.: US 2008/0027001 A1
(43) Pub. Date: Jan. 31, 2008

(54) NOGO RECEPTOR FUNCTIONAL MOTIFS, PEPTIDE MIMETICS, AND MUTATED FUNCTIONAL MOTIFS RELATED THERETO, AND METHODS OF USING THE SAME

(76) Inventors: Andrew Wood, Newtown, PA (US); Alan Katz, Lawrenceville, NJ (US); Ying Gao, East Brunswick, NJ (US); Brian G. Bates, Chelmsford, MA (US); Patrick Doherty, Twickenham (GB); Gareth Williams, Ilford (GB)

Correspondence Address: FITZPATRICK CELLA HARPER & SCINTO, 30 ROCKEFELLER PLAZA, NEW YORK, NY, 10112, US

(21) Appl. No.: 11/774,940

(22) Filed: Jul. 9, 2007

Related U.S. Application Data
(60) Provisional application No. 60/819,086, filed on Jul. 7, 2006.

(51) Int. Cl. A61K 38/00; C07D 211/06; C07D 211/72; C07D 215/00; C07D 277/62; C07D 401/00; C07K 14/00; C07K 16/18; C07K 7/00; C12N 5/06; C40B 30/04; G01N 33/566

(52) U.S. Cl. 514/12; 435/375; 436/501; 506/9; 530/331; 530/350; 530/387.9; 544/324; 546/152; 546/201; 546/226; 546/308; 548/179

(57) ABSTRACT

The present invention provides novel isolated and purified polynucleotides and polypeptides related to functional motifs of the Nogo receptor 1 (NgR1) (e.g., the binding pocket on the side surface of NgR1, functional motifs comprising the amino acid sequence of FRG, etc.) and use of peptides mimicking these functional motifs as antagonists to NgR1 ligands, e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1, etc. The invention also provides antibodies to the mimetic peptide antagonists. The present invention is further directed to novel therapeutics and therapeutic targets and to methods of screening and assessing test compounds for treatments requiring axonal regeneration, i.e., reversal of the effects of NgR1 ligand binding to the NgR1 (i.e., producing inhibition of axonal growth). The present invention also is directed to novel methods for treating disorders arising from inhibition of axonal growth mediated by the binding of NgR1 ligands to the NgR1. Further, the invention is directed to methods of treating a subject with a neurodegenerative disorder, including, but not limited to, Parkinson's disease, Alzheimer's disease, progressive supranuclear palsy, multiple sclerosis, multiple system atrophy, corticobasal degeneration, Huntington's disease, dementia with Lewy bodies, spinocerebellar ataxia, stroke, spinal cord trauma, traumatic brain injury, multiinfarct dementia, epilepsy, and senile dementia, comprising, e.g., antagonizing NgR1.

### RELATED APPLICATIONS

This application claims the benefit of priority from U.S. Provisional Patent Application No. 60/819,086, filed Jul. 7, 2006, the content of which is hereby incorporated by reference herein in its entirety.

### BACKGROUND OF THE INVENTION

1. Field of the Invention

The invention relates to functional motifs of the Nogo receptor 1 (NgR1), e.g., ligand binding site(s) of NgR1 ligands (e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1), peptide mimetics and mutated functional motifs related thereto, all of which may be used in methods of treating, ameliorating, preventing, diagnosing, prognosing, or monitoring disorders arising from inhibition of axonal growth mediated by the binding of NgR1 ligands to the NgR1 (e.g., methods of antagonizing (e.g., reversing, decreasing, reducing, preventing, etc.) axonal growth inhibition mediated by such NgR1 ligands (e.g., methods of treating subjects in need of axonal regeneration), methods of screening for and identifying compounds that may also act as antagonists to NgR1 ligands (e.g., antagonists to ligand binding site(s) of NgR1 ligands (e.g., antagonists to NgR1 functional motifs))) to accomplish the reversal of such inhibition, and antagonistic compounds identified using the peptide mimetics, mutated functional motifs, and methods provided herein.

2. Related Background Art

The central nervous system shows very limited repair after injury; this has been postulated to be due, at least in part, to the presence of inhibitory products associated with damaged central nervous system myelin that prevent axonal regeneration (Berry (1982) Bibl. Anat. 23:1-11). Early studies in this area identified two protein fractions (Caroni and Schwab (1988) J. Cell Biol. 106(4):1281-88) and demonstrated that an antibody raised against these fractions could neutralize the nonpermissive substrate properties of myelin (Caroni and Schwab (1988) Neuron 1(1):85-96).

To date, three myelin molecules have been reported to be inhibitors of axonal growth: (1) the myelin-associated glycoprotein (MAG) (McKerracher et al. (1994) Neuron 13(4):805-11; Mukhopadhyay et al. (1994) Neuron 13(3):757-67); (2) Nogo (e.g., Nogo-A (e.g., the 66-residue extracellular domain of Nogo-A (Nogo-66))) (Chen et al. (2000) Nature 403:434-39; GrandPre et al. (2000) Nature 403:439-44; Prinjha et al. (2000) Nature 403:383-84); and (3) the oligodendrocyte myelin glycoprotein (Wang et al. (2002) Nature 417:941-44). A receptor complex in neurons containing the Nogo receptor 1 (NgR1) (Domeniconi et al. (2002) Neuron 35(2):283-90; Fournier et al. (2001) Nature 409:341-46; Liu et al. (2002) Science 297:1190-93; Wang et al. (2002) Nature 471:941-44;), the low affinity p75 neurotrophin receptor (p75NTR) (Wang et al. (2002) Nature 420:74-78; Wong et al. (2002) Nat. Neurosci. 5(12):1302-08), and Lingo-1 (Mi et al. (2004) Nat. Neurosci. 7(3):221-28; the crystal structure of Lingo-1 is provided by U.S. Patent Application 60/765,443, hereby incorporated by reference herein in its entirety), has been implicated in mediating the response to all three inhibitory molecules. More recently, it has been suggested that for some ligands, NgR2 can substitute for NgR1 (Venkatesh et al. (2005) J. Neurosci. 25:808-22), and that a second TNF receptor superfamily member (member 19; also known as TAJ, TRADE, TRAIN, or TROY) can substitute for p75NTR (Shao et al. (2005) Neuron 45(3):353-59; Park et al. (2005) Neuron 45(3):345-51 (Erratum in He et al. (2005) Neuron 45:815)). Importantly, binding to the receptor complex is required for each inhibitor to mediate inhibitory activity. This redundancy of function may explain disappointing results reported in an NgR1 knockout mouse that cast some doubts on the importance of the receptor as a therapeutic target, at least in spinal injury models (Zheng et al. (2005) Proc. Natl. Acad. Sci. U.S.A. 102(4):1205-10).

MAG can inhibit axonal growth when it is expressed in cells, myelin bound, or presented to neurons as a naturally occurring soluble form (McKerracher et al. (1994) supra; Mukhopadhyay et al. (1994) supra; Tang et al. (1997) Mol. Cell. Neurosci. 9:333-46). MAG appears to have two binding sites, a sialic acid binding site at arginine 118 in Ig domain 1 and a second “inhibitory” site which is absent from the first three Ig domains (Tang et al. (1997a) J. Cell. Biol. 138:1355-66). Soluble MAG does not inhibit neurite outgrowth from neurons that have had terminal sialic acids removed from glycoconjugates by neuraminidase treatment (DeBellard et al. (1996) Mol. Cell. Neurosci. 7:89-101). Soluble MAG binding to the NgR1 and NgR2 is also dependent on sialic acid (Venkatesh et al. (2005) supra). Thus, it would appear that the sialic acid binding site of MAG most probably recognizes the receptor complex via sialic acid-containing glycoconjugates. This site is only required for MAG function when MAG acts as a soluble ligand, as substrate-bound MAG appears to be able to function independently of the sialic acid binding site (Tang et al. (1997a) supra).

MAG belongs to the Siglec (sialic acid-binding Ig-like lectin) family that can bind terminal α2,3-sialic acids on proteins and gangliosides, including GD1a and GT1b (Collins et al. (1997) J. Biol. Chem. 272:1248-55; Collins et al. (1997a) J. Biol. Chem. 272:16889-95; Crocker and Varki (2001) Trends Immunol. 22:337-42: Vyas and Schnaar (2001) Biochemie 83:677-82). It is well established that gangliosides are functional neuronal binding partners for soluble MAG (Vyas et al. (2002) Proc. Natl. Acad. Sci. U.S.A. 99:8412-17; Fujitani et al. (2005) J. Neurochem. 94:15-21). Antibodies that cluster neuronal gangliosides inhibit neurite outgrowth in a manner that is not obviously different from soluble MAG, presumably by coclustering and activating an inhibitory receptor complex on neurons (Vyas et al. (2002) supra; Fujitani et al. (2005) supra; Vinson et al. (2001) J. Biol. Chem. 276:20280-85; Williams et al. (2005) J. Biol. Chem. 280:5862-69). Like the response to MAG, the response to clustered gangliosides is associated with p75NTR function and requires activation of RhoA (Fujitani et al. (2005) supra; Vinson et al. (2001) supra). One explanation for these data is that gangliosides directly interact with one or more components of the NgR1 complex, and thereby function as coreceptors for soluble MAG. In this model, antibodies to gangliosides would inhibit axonal growth by clustering the same NgR1/p75NTR/Lingo-1 complex as MAG.

Two groups have recently solved the crystal structure of the NgR1 (Barton et al. (2003) EMBO J. 23:3291-02; He et al. (2003) Neuron 38:177-85). The receptor has a prominent leucine-rich repeat (LRR) domain, which is composed of amino and carboxy terminal LRR modules that cap nine highly homologous LRR modules. Extensive mutagenesis data has mapped the major sites for binding of all three myelin ligands to the concave face of the LRR domain on the receptor (Lauren et al. (2007) J. Biol. Chem. 282:5715-25). Although immunoprecipitation of GT1b results in the coprecipitation of p75NTR (Yamashita et al. (2002) J. Cell. Biol. 157:565-70), and presumably the other members of the inhibitory complex, nothing is yet known about how gangliosides interact with the three established components of this receptor complex. In this context, the terminal sialic acid on gangliosides interacts with a highly conserved FRG motif in MAG (Tang et al. (1997a) supra) and up to three highly conserved FRG motifs have been observed in the NgR family.

Agents that interfere with the interaction of one or more NgR1 ligands (which may also be an axonal growth inhibitor(s)) with the NgR1 and/or the formation of the higher order receptor-signaling complex may have therapeutic potential and/or be useful biological tools, e.g., for antagonizing (e.g., reversing, decreasing, reducing, preventing, etc.) NgR1 ligand-mediated inhibition of axonal growth. In this context, if small functional motifs could be identified on the NgR1, biologically active peptide mimetics could be developed as specific antagonists, or serve as useful tools in the drug discovery process (see generally, e.g., Hruby (2002) Nat. Rev. Drug Discov. 1(11):847-58).

The invention disclosed herein addresses this problem using analytical ultracentrifugation sedimentation to demonstrate that GT1b can form higher order complexes with the NgR1. This requires the presence of terminal α2-3 sialic acid on the ganglioside, and is inhibited by mutation of the FRG motifs in the receptor. One of the FRG motifs is found within an exposed carboxy-terminal loop of the receptor that lends itself well to the design of a cyclic peptide mimetic. In fact, the inventors showed that a cyclic peptide mimetic of this loop completely prevented GT1b antibodies from inhibiting neurite outgrowth. The same peptide also antagonized the inhibitory response stimulated by soluble MAG, and alanine scanning within the peptide identified the FRG sequence as the functional motif. The inventors have also demonstrated herein that mutations within this motif significantly inhibit soluble MAG from binding to the full-length NgR expressed in cells. FRG peptides may affect MAG function directly or indirectly by interfering with ganglioside interactions with the NgR1-signaling complex.

### SUMMARY OF THE INVENTION

The present invention is based on the identification of functional motifs within the Nogo receptor 1 (NgR1). The invention is also based on the use of peptides mimicking such functional motifs to antagonize NgR1 ligands (NgR1L), which are also axonal growth inhibitors (e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1, etc.). In one embodiment, a putative and/or actual functional motif of the NgR1 has and/or consists essentially of an amino acid sequence selected from the group consisting of YNEPKVT (SEQ ID NOs:2 and 8), LQKFRGSS (SEQ ID NOs:14 and 16), SLPQRLA (SEQ ID NO:4), NLPQRLA (SEQ ID NO:10) and AGRDLKR (SEQ ID NOs:6 and 12). In another embodiment of the invention, a peptide mimetic of a putative and/or actual functional motif of the NgR1 of the invention is provided as an antagonist to one or more NgR1 ligand(s) (NgR1L), i.e., an antagonist to at least one NgR1L. For example, the invention provides an antagonist to an NgR1L (i.e., an antagonist to at least one NgR1L) comprising a polypeptide comprising an amino acid sequence selected from the group consisting of the amino acid sequence of YNEPKVT (SEQ ID NOs:2 and 8), LQKFRGSS (SEQ ID NOs:14 and 16), SLPQRLA (SEQ ID NO:4), NLPQRLA (SEQ ID NO:10), AGRDLKR (SEQ ID NOs:6 and 12), and the amino acid sequences of active fragments thereof.

In one embodiment, the invention provides an antagonist to an NgR1 ligand comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments of the invention, an antagonist to an NgR1 ligand comprises a polypeptide comprising an amino acid sequence selected from the group consisting of the amino acid sequences LQKFRGSS (SEQ ID NOs:14 and 16), KFRGS (SEQ ID NOs:18 and 20), and QKFRG (SEQ ID NOs:22 and 24). In other embodiments, an antagonist of the invention is acetylated and/or amide blocked. In other embodiments, an antagonist of the invention is cyclized (e.g., via homodetic cyclization or a disulfide bond). For example, in one embodiment, the invention provides an antagonist to an NgR1L comprising a polypeptide comprising the amino acid sequence KFRG (SEQ ID NO:26), wherein the polypeptide is cyclized, e.g., by homodetic cyclization, which is a form of cyclization in which the ring consists solely of amino acid residues in eupeptide linkage. In another embodiment, the antagonist comprises at least one D-amino acid. In another embodiment, the antagonist comprises the amino acid sequence of SGRFKQ (SEQ ID NO:37; alternate representation of an antagonist of the invention comprising a homodetic cyclic polypeptide (c[ ]) comprising the amino acid sequence of SEQ ID NO:37 with D-type normative amino acids (lower case letters), i.e., c[sGrfkq]), or an active fragment(s) thereof.

In other embodiments, an antagonist of the invention is cyclized by means of a disulfide bond. In one embodiment, the invention provides a cyclized antagonist to an NgR1 ligand comprising a polypeptide comprising an amino acid sequence selected from the group consisting of the amino acid sequence of SEQ ID NO:31, the amino acid sequence of SEQ ID NO:32, the amino acid sequence of SEQ ID NO:33, the amino acid sequence of SEQ ID NO:34, and the amino acid sequences of active fragments thereof. In one embodiment, the invention provides an antagonist of at least one NgR1 ligand comprising a polypeptide comprising the amino acid sequence of CLQKFRGSSC (SEQ ID NO:31). In another embodiment, the antagonist comprises a polypeptide comprising the amino acid sequence of CKFRGSC (SEQ ID NO:32). In another embodiment, the antagonist comprises a polypeptide comprising the amino acid sequence of CQKFRGC (SEQ ID NO:33). In another embodiment, the antagonist comprises a polypeptide comprising the amino acid sequence of CKFRGC (SEQ ID NO:34). In several embodiments, an antagonist of the invention comprises at least one D-amino acid. In other embodiments, an antagonist of the invention is acetylated and/or amide blocked. In another embodiment, the antagonists described above antagonize an NgR1 binding fragment of an NgR1 ligand selected from the group consisting of myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1.

The invention also provides methods of using the antagonists of the invention, e.g., methods of screening for other antagonists (e.g., test compounds), and methods of antagonizing NgR1 ligand-mediated inhibition of axonal growth in a sample or subject (e.g., a human subject). In one embodiment, the invention provides a method of screening for compounds that antagonize NgR1 ligands comprising the steps of contacting a sample containing an NgR1 ligand and an antagonist of the invention with the compound; and determining whether the interaction between the NgR1 ligand and the antagonist of the invention in the sample is decreased relative to the interaction of the NgR1 ligand and the antagonist of the invention in a sample not contacted with the compound, whereby a decrease in the interaction of the NgR1 ligand and the antagonist of the invention in the sample contacted with the compound identifies the compound as one that competes with the antagonist of the invention. In some embodiments of these methods, the antagonist comprises a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. Additionally, in some embodiments, the compound is further identified as one that antagonizes at least one NgR1 ligand.

The invention also provides a method of antagonizing inhibition of axonal growth mediated by an NgR1 ligand in a sample comprising the step of contacting the sample with an antagonist of the invention. In one embodiment, the antagonist to the at least one NgR1 ligand is a peptide that mimics a functional motif of the NgR1. The invention also provides a method of antagonizing inhibition of axonal growth in a sample comprising the step of contacting the sample with an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments, the inhibition of axonal growth is mediated by at least one NgR1 ligand. In some embodiments of the invention, the antagonizing of inhibition of axonal growth results in regeneration of axons.

In one embodiment, the invention provides a method of regenerating axons and/or antagonizing inhibition of axonal growth in a subject (e.g., a human subject) comprising administering to the subject an antagonist of the invention. For example, the invention provides a method of antagonizing inhibition of axonal growth in a subject comprising the step of administering to the subject an effective amount of an antagonist to at least one NgR1 ligand, e.g., wherein the antagonist to the at least one NgR1 ligand is a peptide that mimics a functional motif of the NgR1. In another embodiment, the invention provides a method of antagonizing inhibition of axonal growth in a subject comprising the step of administering to the subject an effective amount of an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments, the inhibition of axonal growth is mediated by at least one NgR1 ligand. In some embodiments, the antagonizing of inhibition of axonal growth results in regeneration of axons. In other embodiments, the method of regenerating axons and/or antagonizing inhibition of axonal growth in a subject comprises administering to the subject an antagonist of the invention, wherein the subject has suffered an injury to the central nervous system, e.g., wherein the subject has suffered from a stroke and/or some other form of traumatic brain and/or spinal cord injury, etc. In another embodiment, the subject suffers from, or has suffered from, a neuronal degenerative disease, e.g., multiple sclerosis, Parkinson's disease, Alzheimer's disease, etc.

In addition, the present invention provides pharmaceutical compositions comprising an antagonist of the invention, and routes of administration of such a composition, for use in the methods of the invention. In some embodiments, a pharmaceutical composition of the invention comprises a pharmaceutically acceptable carrier and an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

The invention also provides an antagonist to an NgR1 ligand comprising a polypeptide comprising an amino acid sequence selected from the group consisting of the amino acid sequence of SEQ ID NO:2, the amino acid sequence of SEQ ID NO:4, the amino acid sequence of SEQ ID NO:6, the amino acid sequence of SEQ ID NO:10, and the amino acid sequences of active fragments thereof. In some embodiments, the polypeptide is cyclized (e.g. via a disulfide bond, etc.).

The invention also provides an isolated antibody capable of specifically binding to a polypeptide comprising an amino acid sequence selected from the group consisting of the amino acid sequences of SEQ ID NOs:2, 4, 6, 8, 10, 12, 14, 16, 18, 20, 22, 24, 26, 27, 28, 29, 30, 31, 32, 33, 34, 37, and the amino acid sequences of active fragments thereof. In some embodiments, the antibody is produced in response to an immunogen comprising an antagonist to at least one NgR1 ligand. Also provided is an isolated antibody capable of specifically binding to an antagonist to at least one NgR1 ligand.

In at least one embodiment, the invention provides an NgR1 functional motif comprising the amino acid sequence FRG. In other embodiments, without limitation, the functional motif is located on loop 2 of NgR1; the functional motif binds GT1b; and/or the functional motif binds MAG. In other embodiments, the invention provides an antagonist(s) to such an NgR1 functional motif(s). In other embodiments, such an antagonist is selected from the group consisting of WAY-100080, WY-48185, WY-23626, CL-391991, CL-306115, and WY-46543.

In another embodiment, the invention provides a method of determining whether a compound inhibits an NgR1 ligand from binding NgR1 comprising the steps of contacting a sample containing an NgR1 ligand and NgR1 with a test compound; and determining whether the interaction between the NgR1 ligand and NgR1 is decreased relative to the interaction of the NgR1 ligand and NgR1 in a sample not contacted with the compound, wherein a decrease in the interaction of the NgR1 ligand and NgR1 in the sample contacted with the compound identifies the compound as one that inhibits an NgR1 ligand from binding NgR1. In another embodiment, the NgR1 is expressed on the surface of at least one cell (e.g., a CHO cell; a COS-7 cell, etc.). In other embodiments, the NgR1 ligand is, without limitation, MAG; MAG-Fc; MAG-AP; p75NTR; and/or Nogo-66-AP. In other embodiments, the NgR1 ligand is expressed on the surface of at least one cell (e.g., a CHO cell; a COS-7 cell, etc.). In other embodiments, the NgR1 is fused to alkaline phosphatase (AP). In other embodiments, the invention provides a cell expressing cell surface p75NTR. In other embodiments, the invention provides a cell expressing NgR-AP.

In another embodiment, the invention provides a method of identifying an NgR1 ligand antagonist comprising the step of screening, e.g., a database of compounds for at least one compound that mimics an NgR1 functional motif. In another embodiment, the method further comprises, after the step of screening, e.g., a database, the step of determining whether the at least one compound that mimics an NgR1 functional motif inhibits an NgR1 ligand from binding NgR1. In a further embodiment, the step of determining comprises the aforementioned method of determining whether a compound inhibits an NgR1 ligand from binding NgR1. The invention further provides such NgR1 ligand antagonist(s) identified by such methods. In another embodiment, the invention provides a method of identifying an NgR1 ligand antagonist comprising the step of screening, e.g., a database of compounds for at least one compound that binds an NgR1 functional motif. In another embodiment, the method further comprises, after the step of screening, e.g., a database, the step of determining whether the at least one compound that binds an NgR1 functional motif inhibits an NgR1 ligand from binding NgR1. In a further embodiment, the step of determining comprises the aforementioned method of determining whether a compound inhibits an NgR1 ligand from binding NgR1. The invention further provides such NgR1 ligand antagonist(s) identified by such methods. In other embodiments, the step of screening comprises using PharmDock.

In other embodiments, the invention provides a method of treating a subject with a disorder arising from the inhibition of axonal growth mediated by the binding of an NgR1 ligand to the NgR1 comprising administering to the subject an antagonist of the invention. In other embodiments, the antagonist is selected from the group consisting of WAY-100080, WY-48185, WY-23626, CL-391991, CL-306115, and WY-46543.

In other embodiments, the invention provides a binding pocket of NgR1, wherein the binding pocket is on the side surface of NgR1. In other embodiments, the binding pocket further comprises the amino acid sequence of FRG. In other embodiments, the amino acid sequence of FRG is further defined as F278, R279, and G280.

In other embodiments, the invention provides a method of treating a subject with a neurodegenerative disorder comprising the step of antagonizing NgR1. In other embodiments, the step of antagonizing NgR1 comprises inhibiting an NgR1 ligand from binding NgR1. In other embodiments, the step of antagonizing NgR1 comprises administering to the subject an antagonist of NgR1. In further embodiments, the antagonist of NgR1 is an antagonist of the invention. In other embodiments, the antagonist of NgR1 is selected from the group consisting of a peptide antagonist and a small molecule antagonist. In further embodiments, the small molecule antagonist is selected from the group consisting of WAY-100080, WY-48185, WY-23626, CL-391991, CL-306115, and WY-46543. In other embodiments, the neurodegenerative disorder is selected form the group consisting of Parkinson's disease, Alzheimer's disease, progressive supranuclear palsy, multiple sclerosis, multiple system atrophy, corticobasal degeneration, Huntington's disease, dementia with Lewy bodies (Lewy body dementia), spinocerebellar ataxia, stroke, spinal cord trauma, traumatic brain injury, multiinfarct dementia, epilepsy, senile dementia, Alexander disease, Alper's disease, amyotrophic lateral sclerosis, ataxia telangiectasia, Batten disease (Spielmeyer-Vogt-Sjogren-Batten disease), bovine spongiform encephalopathy, Canavan disease, Cockayne syndrome, Creutzfeldt-Jakob disease, HIV-associated dementia, Kennedy's disease, Krabbe disease, Machado-Joseph disease (spinocerebellar ataxia type 3), neuroborreliosis, Pelizaeus-Merzbacher disease, Pick's disease, primary lateral sclerosis, prion diseases, Refsum's disease, Sandhoff disease, Schilder's disease, schizophrenia, spinal muscular atrophy, Steele-Richardson-Olszewski disease, and tabes dorsalis. In other embodiments, the present invention provides methods of treatment, etc. related to peripheral neuropathies, including, but not limited to, distal axonopathies, myelinopathies, and neuronopathies. In other embodiments, the methods of treating of the invention may also alleviate symptoms associated with neurodegenerative disorders and peripheral neuropathies including, but not limited to, pain.

The present invention also provides kits comprising an antagonist of the invention to aid in practicing the methods disclosed herein.

### BRIEF DESCRIPTION OF THE DRAWINGS

FIG. 1A shows the concave face of NgR1 in space-filled mode; the residues critical for binding to ligand (including, but not limited to, myelin-associated glycoprotein, oligodendrocyte myelin glycoprotein, Nogo-A, Nogo-66, etc.) are shown by dark patches, which are predicted to correspond to the dominant cluster of energy minima surrounding the protein (derived by the statistical potential field, and shown as a collection of spheres in near-perfect alignment with the critical binding residues localized within the dark patches).

FIG. 1B shows the convex face of NgR1 in space-filled mode; the two actual and/or putative ligand binding sites are denoted by rectangles, which are predicted to correspond to the clusters of energy minima for a simple 3.55 Å diameter van der Waals probe that define two small pockets within the area enclosed by the rectangles (in proximity to the shown spheres). The three occurrences of the FRG motif are also shown in FIG. 1B, denoted as ovals, with the 198FRG200 and 278FRG280 peptides shown as neighboring the predicted small molecule binding pockets (denoted by the two rectangles). A ribbon diagram of the Nogo receptor 1 (NgR1), denoting the four putative and/or actual functional motifs (darkened portions of the ribbon, with corresponding sequences indicated), is shown in FIG. 1C.

The relative fluorescence units (RFU (x1000); y-axis) of increasing concentrations of MAG-AP (MAG-AP, μg/ml; x-axis) binding to parental (control) CHO cells (CHO; diamonds) or CHO cells stably expressing NgR1 (NgR1/CHO; circles) in the absence (−; solid lines) or presence (+; dashed lines) of neuraminidase are shown in FIG. 2A. The relative binding to NgR1 (y-axis) of 10 μg/ml MAG-AP (diamonds) or 10 μg/ml Nogo66-AP (circles) in increasing concentrations of neuraminidase (mU/ml; x-axis) is shown in FIG. 2B.

Shown in FIG. 3 are sedimentation coefficient distribution (c(S)) plots of NgR(310)-fc as a function of increasing GT1b (FIG. 3A), GM1 (FIG. 3B), and asialo-GM1 (aGM1) (FIG. 3C). The effects of GT1b (22 μM) on sedimentation of NgR1 constructs containing single point mutations was also determined and shown for mutants R279E (FIG. 3D), R151E (FIG. 3E), and R199E (FIG. 3F).

Results from 3 independent experiments were pooled to obtain the mean length of the longest cerebellar neurite (μm; y-axis)±SEM (bars) from 100-120 neurons cultured over monolayers of established 3T3 cells in media alone (control; black bars) or media containing 20 μg/ml anti-GT1b antibody (+GT1b @20 μg/ml; white bars) for different treatment times (x-axis), as shown in FIG. 4A. As shown in FIG. 4B, results from between 3 and 13 independent experiments [as noted in the parentheses] were pooled to obtain the mean length of the longest cerebellar neurite (μm; y-axis)±SEM (bars) from 100-120 neurons cultured over monolayers of established 3T3 cells in media containing 0-40 μg/ml anti-GT1b antibody in the absence (filled circles) or presence (open circles) of the NRL2 peptide (N-Ac-CLQKFRGSSC-NH₂ (SEQ ID NO:31)) at 100 μg/ml.

Results from between 3 and 13 independent experiments [as noted in the parentheses] were pooled to obtain the mean length of the longest cerebellar neurite (μm; y-axis)±SEM (bars) from 100-120 neurons cultured over monolayers of established 3T3 cells in media supplemented for 23-27 hr without MAG-Fc (white columns) or with MAG-Fc at 25 μg/ml (cross-hatched columns) in the absence (control) or presence of 100 μg/ml NRL peptides 1-4 (x-axis), as shown in FIG. 5A. As shown in FIG. 5B, results from between 3 and 13 independent experiments [as noted in the parentheses] were pooled to obtain the mean length of the longest cerebellar neurite (μm; y-axis)±SEM (bars) from 120-150 neurons cultured over monolayers of established 3T3 cells in control media (filled circles) or media supplemented with the MAG-Fc at 25 μg/ml (open circles) in the presence of the artificially cyclized, acetylated, and amide-blocked NRL2 peptide (N-Ac-CLQKFRGSSC-NH₂ (SEQ ID NO:31)) at the given concentrations (x-axis).

The mean lengths of the longest neurite (μm; y-axis)±SEM (bars) from about 100-120 neurons of 3 to 5 independent cultures of cerebellar neurons over monolayers of established 3T3 cells in media supplemented with 20 μg/ml MAG-Fc alone (0 μg/ml peptide) or in the presence of increasing concentrations (μg/ml; x-axis) of NRL2a (N-Ac-CKFRGSC-NH₂ (SEQ ID NO:32); filled circles) or NRL2b (N-Ac-CQKFRGC-NH₂ (SEQ ID NO:33); open circles) are shown in FIG. 6A. Results from between 3 and 4 independent experiments [as noted in the parentheses] were pooled to obtain the mean length of the longest cerebellar neurite (μm; y-axis)±SEM (bars) from 100-120 neurons cultured over monolayers of established 3T3 cells in media supplemented for 23-27 hr without MAG-Fc (black columns) or with MAG-Fc at 20 μg/ml (white columns) in the absence (no pep) or presence of 100 μg/ml NRL2b (N-Ac-CQKFRGC-NH₂ SEQ ID NO:33), NRL2bA1 (A1; N-Ac-CQAFRGC-NH₂: SEQ ID NO:46), NRL2bA2 (A2; N-Ac-CQKARGC-NH₂; SEQ ID NO:47), NRL2bA3 (A3; N-Ac-CQKFAGC-NH₂; SEQ ID NO:48), NRL2bA4 (A4; N-Ac-CQKFRAC-NH₂; SEQ ID NO:49) or linear NRL2b (LNRL2b; QKFRG; SEQ ID NO: 22) peptides (x-axis), as shown in FIG. 6B. The mean lengths of the longest neurite (μm; y-axis)±SEM (bars) from about 100-120 neurons of 3 independent cultures of cerebellar neurons over monolayers of established 3T3 cells in control media (filled circles) or media supplemented with 20 μg/ml MAG-Fc (open circles) are presented, both with increasing concentrations (μg/ml; x-axis) of either NRL2bA1 (CQAFRGC; SEQ ID NO:46) as shown in FIG. 6C, or NRL2bA2 (CQKARGC; SEQ ID NO:47) as shown in FIG. 6D. The mean lengths of the longest neurite (μm; y-axis)±SEM (bars) from about 100-120 neurons of 2 independent cultures of cerebellar neurons over monolayers of established 3T3 cells in control media (filled circles) or media supplemented with 20 μg/ml MAG-Fc (open circles), both with increasing concentrations (μg/ml; x-axis) of hriNRL2 (N-Ac-c[sGrfkq]-NH₂ (SEQ ID NO:37)) are shown in FIG. 6E. Shown in FIG. 6F are the neurite lengths (neurite length, % of control; y-axis)±SEM (bars) from about 100-120 neurons of 2 independent cultures of cerebellar neurons over monolayers of established 3T3 cell in media supplemented with 20 μg/ml MAG-Fc (open circles) a percentage of the neurite lengths of cultures in control media (filled circles), both with increasing concentrations (μg/ml; x-axis) of hriNRL2 (N-Ac-c[sGrfkq]-NH₂ (SEQ ID NO:37)).

Shown in FIG. 7A are Western blot analyses (WB) with either antibodies to NgR1 (upper panel; NgR) or p75NTR (lower panel; p75) of lysates isolated from CHO-K1 cells transfected with (+) or without (−) p75NTR (p75) and/or vector alone, wild type NgR1 (WT), mutant NgR1EM7 (K277D/R279D), mutant NgR1EM8 (K277A/R279A), mutant NgR1EM10 (K277A), or mutant NgR1EM11 (R279A) and immunoprecipitated (IP) with goat anti-human NgR1 antibody. The relative binding (y-axis) of wild type NgR1 (WT) or one of the following four mutant NgR1 (EM7 (K277D, R279D); EM8 (K277A, R279A); EM10 (K277A); or EM11 (R279A)) to alkaline phosphatase-labeled MAG (MAG-AP) or alkaline phosphatase-labeled Nogo-66 (Nogo66-AP) is shown in FIG. 7B. The percent binding (% of binding to p75; y-axis)±SEM (bars), from four independent experiments, of p75NTR to lysates isolated from cells transfected with vector alone, wild type NgR1 (WT), mutant NgR1EM7 (K277D/R279D), mutant NgR1EM8 (K277A/R279A), mutant NgR1EM10 (K277A), or mutant NgR1EM11 (R279A) and immunoprecipitated with anti-NgR1 antibody is shown in FIG. 7C.

The hydrophobic feature of the side surface of NgR1 is shown in FIG. 8.

The convergence of a functionally validated NRL2 peptide site and side-surface binding pocket of NgR1 is shown in FIG. 9.

Exemplary lead compounds (WAY-100080 (see, e.g., Patent No. GB 2044254); WY-48185 (see, e.g., Patent No. GB 2183641 A1); WY-23626 (see, e.g., Patent No. DE 2144080); CL-391991 (purchased from Maybridge, Cornwall, UK); CL-306115 (see, e.g., Patent No. EP 233461); and WY-46543 (see, e.g., U.S. Pat. No. 4,554,355)) identified by PharmDock screening of the side-surface binding pocket of NgR1 that promote neurite outgrowth within a MAG-Fc inhibitory environment are shown in FIG. 10.

Shown in FIG. 11 is a schematic of the pSMED2 expression vector comprising nucleotides encoding wild type NgR(310)-fc.

### DETAILED DESCRIPTION OF THE INVENTION

The limitations presented by conventional deletion analysis were overcome by adopting a rational approach to identify putative and/or actual functional motifs in the Nogo receptor 1 (NgR1) (see Example 2.1). Based on this approach, three independent small-constrained peptides that mimic an exposed loop at the carboxy terminal region of the LRR structure of the NgR1 were identified. These peptides can act as antagonists to NgR1 ligands, (e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1), i.e., can act to antagonize (e.g., reverse, decrease, reduce, prevent, etc.) the biological consequences of an NgR1 ligand(s) binding to the NgR1 complex in neurons (e.g., inhibition of axonal growth (Examples 2.5 and 2.6) and/or the formation of the higher order receptor-signaling complex). In addition, alanine scanning of one of the peptides points to an FRG motif as being the key functional motif within the exposed loop, and mutations within this loop were found to inhibit MAG binding to the NgR1. As such, the invention provides polynucleotides and polypeptides related to the putative and/or actual functional motifs and/or mimetic peptide antagonists, including the mimetic peptide antagonists resulting from mutations used for alanine scanning.

Polynucleotides and Polypeptides

The present invention provides novel isolated and purified polynucleotides and polypeptides homologous to putative and/or actual functional domains of the Nogo receptor 1 (NgR1). It is part of the invention that peptide mimetics to putative and/or actual functional domains of the NgR1 may be used as antagonists to NgR1 ligands, i.e., to inhibit the biological effect of NgR1 ligand binding to the NgR1.

For example, the invention provides purified and isolated polynucleotides encoding three putative NgR1 functional motifs, which may function as NgR1 ligand antagonists, herein designated “NRL1,” “NRL3,” and “NRL4.” Preferred DNA sequences of the invention include genomic and cDNA sequences and chemically synthesized DNA sequences.

The nucleotide sequences of cDNAs encoding human NRL1 (hNRL1), human NRL3 (hNRL3), and human NRL4 (hNRL4), designated human cDNA, are set forth in SEQ ID NOs:1, 3, and 5, respectively. Polynucleotides of the present invention also include polynucleotides that hybridize under stringent conditions to SEQ ID NOs:1, 3, or 5, or complements thereof, and/or encode polypeptides that retain substantial biological activity of hNRL1, hNRL3, or hNRL4, respectively. Polynucleotides of the present invention also include continuous portions of the sequences set forth in SEQ ID NOs:1, 3, or 5 comprising at least 12 consecutive nucleotides.

The amino acid sequences of hNRL1, hNRL3, and hNRL4 are set forth in SEQ ID NOs:2, 4, and 6, respectively. Polypeptides of the present invention also include continuous portions of any of the sequences set forth in SEQ ID NOs:2, 4, and 6, comprising at least 4 consecutive amino acids. Polypeptides of the invention also include any of the sequences set forth in SEQ ID NOs:2, 4, and 6, including continuous portions thereof, wherein one or more of the L-amino acids are replaced with their corresponding D-amino acids. Polypeptides of the present invention also include any continuous portion of any of the sequences set forth in SEQ ID NO:2, 4, and 6 that retains substantial biological activity (i.e., an active fragment) of full-length human hNRL1, hNRL3, and hNRL4, respectively. Additionally, a polypeptide of the invention may be acetylated and/or amide blocked using well-known methods. Polynucleotides of the present invention also include, in addition to those polynucleotides of human origin described above, polynucleotides that encode any of the amino acid sequences set forth in SEQ ID NO:2, 4, or 6, or continuous portions thereof (e.g., active fragments thereof), and that differ from the polynucleotides of human origin described above only due to the well-known degeneracy of the genetic code.

The nucleotide sequences of cDNAs encoding rat NRL1 (rNRL1), rat NRL3 (rNRL3), and rat NRL4 (rNRL4), designated rat cDNA, are set forth in SEQ ID NOs:7, 9, and 11, respectively. Polynucleotides of the present invention also include polynucleotides that hybridize under stringent conditions to SEQ ID NOs:7, 9, or, 11, or complements thereof, and/or encode polypeptides that retain substantial biological activity of rNRL1, rNRL3, or rNRL4, respectively. Polynucleotides of the present invention also include continuous portions of the sequences set forth in SEQ ID NOs:7, 9, or 11 comprising at least 12 consecutive nucleotides.

The amino acid sequences of rNRL1, rNRL3, and rNRL4 are set forth in SEQ ID NOs:8, 10, and 12, respectively. Polypeptides of the present invention also include continuous portions of any of the sequences set forth in SEQ ID NOs:8, 10, and 12, comprising at least 4 consecutive amino acids. Polypeptides of the invention also include any of the sequences set forth in SEQ ID NOs:8, 10, and 12, including continuous portions thereof, wherein one or more of the L-amino acids are replaced with their corresponding D-amino acids. Polypeptides of the present invention also include any continuous portion of any of the sequences set forth in SEQ ID NOs:8, 10, and 12 that retains substantial biological activity (i.e., an active fragment) of full-length rNRL1, rNRL3, and rNRL4, respectively. Additionally, a polypeptide of the invention may be acetylated and/or amide blocked using well-known methods. Polynucleotides of the present invention also include, in addition to those polynucleotides of rat origin described above, polynucleotides that encode any of the amino acid sequences set forth in SEQ ID NOs:8, 10, and 12, or continuous portions thereof (e.g., active fragments thereof), and that differ from the polynucleotides of rat origin described above only due to the well-known degeneracy of the genetic code.

The invention also provides purified and isolated polynucleotides encoding a novel NgR1 functional motif, which may also be used as a mimetic peptide antagonist to an NgR1 ligand, herein designated “NRL2.” Preferred DNA sequences of the invention include genomic and cDNA sequences and chemically synthesized DNA sequences.

The nucleotide sequence of a cDNA encoding human NRL2 (hNRL2), designated human cDNA, is set forth in SEQ ID NO:13. Polynucleotides of the present invention also include polynucleotides that hybridize under stringent conditions to SEQ ID NO:13, or its complement, and/or encode polypeptides that retain substantial biological activity of hNRL2. Polynucleotides of the present invention also include continuous portions of the sequence set forth in SEQ ID NO:13 comprising at least 12 consecutive nucleotides.

The amino acid sequence of hNRL2 is set forth in SEQ ID NO:14. Polypeptides of the present invention also include continuous portions of the sequence set forth in SEQ ID NO:14 comprising at least 4 consecutive amino acids. Polypeptides of the invention also include the sequence set forth in SEQ ID NO:14, including continuous portions thereof, wherein one or more of the L-amino acids are replaced with their corresponding D-amino acids. Polypeptides of the present invention also include any continuous portion of the sequence set forth in SEQ ID NO:14 that retains substantial biological activity (i.e., an active fragment) of full-length hNRL2, e.g., KFRG (i.e., SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:14 or a continuous portion thereof (e.g., an active fragment thereof), and that differ from the polynucleotides of human origin described above only due to the well-known degeneracy of the genetic code.

The nucleotide sequence of a cDNA encoding rat NRL2 (rNRL2), designated rat cDNA, is set forth in SEQ ID NO:15. Polynucleotides of the present invention also include polynucleotides that hybridize under stringent conditions to SEQ ID NO:15, or its complement, and/or encode polypeptides that retain substantial biological activity of rNRL2. Polynucleotides of the present invention also include continuous portions of the sequence set forth in SEQ ID NO:15 comprising at least 12 consecutive nucleotides.

The amino acid sequence of rNRL2 is set forth in SEQ ID NO:16. Polypeptides of the present invention also include continuous portions of the sequence set forth in SEQ ID NO:16 comprising at least 4 consecutive amino acids. Polypeptides of the invention also include the sequence set forth in SEQ ID NO:16, including continuous portions thereof, wherein one or more of the L-amino acids are replaced with their corresponding D-amino acids. Polypeptides of the present invention also include any continuous portion of the sequence set forth in SEQ ID NO:16 that retains substantial biological activity (i.e., an active fragment) of full-length rNRL2, e.g., KFRG (i.e., SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:16 or a continuous portion thereof (e.g., an active fragment thereof), and that differ from the polynucleotides of rat origin described above only due to the well-known degeneracy of the genetic code.

The invention also provides purified and isolated polynucleotides encoding a novel mimetic peptide antagonist to an NgR1 ligand, herein designated “NRL2a.” Preferred DNA sequences of the invention include genomic and cDNA sequences and chemically synthesized DNA sequences.

The nucleotide sequence of a cDNA encoding human NRL2a (hNRL2a), designated human cDNA, is set forth in SEQ ID NO:17. Polynucleotides of the present invention also include polynucleotides that hybridize under stringent conditions to SEQ ID NO:17, or its complement, and/or encode polypeptides that retain substantial biological activity of hNRL2a. Polynucleotides of the present invention also include continuous portions of the sequence set forth in SEQ ID NO:17 comprising at least 12 consecutive nucleotides.

The amino acid sequence of hNRL2a is set forth in SEQ ID NO:18. Polypeptides of the present invention also include continuous portions of the sequence set forth in SEQ ID NO:18 comprising at least 4 consecutive amino acids. Polypeptides of the invention also include the sequence set forth in SEQ ID NO:18, including continuous portions thereof, wherein one or more of the L-amino acids are replaced with their corresponding D-amino acids. Polypeptides of the present invention also include any continuous portion of the sequence set forth in SEQ ID NO:18 that retains substantial biological activity (i.e., an active fragment) of full-length hNRL2a, e.g., KFRG (SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:18 or a continuous portion thereof (e.g., an active fragment thereof), and that differ from the polynucleotides of human origin described above only due to the well-known degeneracy of the genetic code.

The nucleotide sequence of a cDNA encoding rat NRL2a (rNRL2a), designated rat cDNA, is set forth in SEQ ID NO:19. Polynucleotides of the present invention also include polynucleotides that hybridize under stringent conditions to SEQ ID NO:19, or its complement, and/or encode polypeptides that retain substantial biological activity of rNRL2a. Polynucleotides of the present invention also include continuous portions of the sequence set forth in SEQ ID NO:19 comprising at least 12 consecutive nucleotides.

The amino acid sequence of rNRL2a is set forth in SEQ ID NO:20. Polypeptides of the present invention also include continuous portions of the sequence set forth in SEQ ID NO:20 comprising at least 4 consecutive amino acids. Polypeptides of the invention also include the sequence set forth in SEQ ID NO:20, including continuous portions thereof, wherein one or more of the L-amino acids are replaced with their corresponding D-amino acids. Polypeptides of the present invention also include any continuous portion of the sequence set forth in SEQ ID NO:20 that retains substantial biological activity (i.e., an active fragment) of full-length rNRL2a, e.g., KFRG (SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:20 or a continuous portion thereof, and that differ from the polynucleotides of rat origin described above only due to the well-known degeneracy of the genetic code.

The invention also provides purified and isolated polynucleotides encoding another novel mimetic peptide antagonist to an NgR1 ligand, herein designated “NRL2b.” Preferred DNA sequences of the invention include genomic and cDNA sequences and chemically synthesized DNA sequences.

The nucleotide sequence of a cDNA encoding human NRL2b (hNRL2b), designated human cDNA, is set forth in SEQ ID NO:21. Polynucleotides of the present invention also include polynucleotides that hybridize under stringent conditions to SEQ ID NO:21, or its complement, and/or encode polypeptides that retain substantial biological activity of hNRL2b. Polynucleotides of the present invention also include continuous portions of the sequence set forth in SEQ ID NO:21 comprising at least 12 consecutive nucleotides.

The amino acid sequence of hNRL2b is set forth in SEQ ID NO:22. Polypeptides of the present invention also include continuous portions of the sequence set forth in SEQ ID NO:22 comprising at least 4 consecutive amino acids. Polypeptides of the invention also include the sequence set forth in SEQ ID NO:22, including continuous portions thereof, wherein one or more of the L-amino acids are replaced with their corresponding D-amino acids. Polypeptides of the present invention also include any continuous portion of the sequence set forth in SEQ ID NO:22 that retains substantial biological activity (i.e., an active fragment) of full-length hNRL2b, e.g., KFRG (SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:22 or a continuous portion thereof, and that differ from the polynucleotides of human origin described above only due to the well-known degeneracy of the genetic code.

The nucleotide sequence of a cDNA encoding rat NRL2b (rNRL2b), designated rat cDNA, is set forth in SEQ ID NO:23. Polynucleotides of the present invention also include polynucleotides that hybridize under stringent conditions to SEQ ID NO:23, or its complement, and/or encode polypeptides that retain substantial biological activity of rNRL2b. Polynucleotides of the present invention also include continuous portions of the sequence set forth in SEQ ID NO:23 comprising at least 12 consecutive nucleotides.

The amino acid sequence of rNRL2b is set forth in SEQ ID NO:24. Polypeptides of the present invention also include continuous portions of the sequence set forth in SEQ ID NO:24 comprising at least 4 consecutive amino acids. Polypeptides of the invention also include the sequence set forth in SEQ ID NO:24, including continuous portions thereof, wherein one or more of the L-amino acids are replaced with their corresponding D-amino acids. Polypeptides of the present invention also include any continuous portion of the sequence set forth in SEQ ID NO:24 that retains substantial biological activity (i.e., an active fragment) of full-length rNRL2b, e.g., KFRG (SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:24 or a continuous portion thereof, and that differ from the polynucleotides of rat origin described above only due to the well-known degeneracy of the genetic code.

The invention also provides purified and isolated polynucleotides encoding the novel NgR1 functional motifs and the mimetic peptide antagonists of the invention, e.g., NRL2, NRL2a, and NRL2b, as cyclized mimetic peptides. Preferred DNA sequences of the invention include genomic and cDNA sequences and chemically synthesized DNA sequences. One of skill in the art will recognize that the present invention also includes other cyclized molecules, such as cyclized mimetic peptides based on NRL1, NRL3, and NRL4, etc. Additionally, a polypeptide of the invention may be acetylated and/or amide blocked using well-known methods.

For example, the amino acid sequences of artificially cyclized, acetylated and amide blocked NRL2, NRL2a, and NRL2b are set forth in SEQ ID NOs:31, 32, and 33, respectively. Polypeptides of the present invention also include continuous portions of any of the sequences set forth in SEQ ID NOs:31, 32, or 33, comprising at least 4 consecutive amino acids. Polypeptides of the present invention also include any continuous portion of any of the sequences set forth in SEQ ID NOs:31, 32, or 33 that retains substantial biological activity (i.e., an active fragment) of full-length NRL2, NLR2a, or NRL2b, respectively, e.g., KFRG (SEQ ID NO:26). Another polypeptide of the invention is the artificially cyclized, acetylated, and amide blocked KFRG (SEQ ID NO:34). As other examples, the amino acid sequences of artificially cyclized, acetylated and amide blocked NRL1 (human or rat), human NRL3, rat NRL3, and NRL4 (human or rat) are set forth in SEQ ID NOs:27, 28, 29, and 30, respectively. Polypeptides of the invention also include any of the sequences set forth in SEQ ID NOs:27, 28, 29, 30, 31, 32, 33, or 34, including continuous portions thereof, wherein one or more of the L-amino acids are replaced with their corresponding D-amino acids.

Based on the amino acid sequences provided in SEQ ID NOs:27, 28, 29, 30, 31, 32, 33, or 34, a skilled artisan could determine one or more DNA sequences that would encode for each of such peptides. As such, polynucleotides of the present invention also include polynucleotides (e.g., genomic, cDNA, and chemically synthesized sequences) that encode an amino acid sequence set forth in SEQ ID NOs:27, 28, 29, 30, 31, 32, 33, or 34, or continuous portions thereof.

For example, a nucleotide sequence of that encodes KFRG, is set forth in SEQ ID NO:25. Polynucleotides of the present invention also include polynucleotides that hybridize under stringent conditions to SEQ ID NO:25, or its complement, and/or encode polypeptides that retain substantial biological activity of KFRG. Polynucleotides of the present invention also include continuous portions of the sequence set forth in SEQ ID NO:25 comprising at least 9 consecutive nucleotides.

As described above, the amino acid sequence of KFRG is set forth in SEQ ID NO:26. Polypeptides of the present invention also include continuous portions of the sequence set forth in SEQ ID NO:26 comprising at least 3 consecutive amino acids. Polypeptides of the invention also include the sequence set forth in SEQ ID NO:26, including continuous portions thereof, wherein one or more of the L-amino acids are replaced with their corresponding D-amino acids. Polypeptides of the present invention also include any continuous portion of the sequence set forth in SEQ ID NO:26 that retains substantial biological activity (i.e., an active fragment) of full-length human KFRG, e.g., KFR. Additionally, a polypeptide of the invention may be cyclized, acetylated and/or amide blocked using well-known methods. Polynucleotides of the present invention also include, in addition to those polynucleotides described above, polynucleotides that encode the amino acid sequence set forth in SEQ ID NO:26 or a continuous portion thereof (e.g., an active fragment thereof), and that differ from the polynucleotides described above only due to the well-known degeneracy of the genetic code.

The isolated polynucleotides of the present invention may be used as hybridization probes and primers to identify and isolate nucleic acids having sequences identical to, or similar to, those encoding the disclosed polynucleotides. Hybridization methods for identifying and isolated nucleic acids include polymerase chain reaction (PCR), Southern hybridization, and Northern hybridization, and are well known to those skilled in the art.

Hybridization reactions can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | | Hybridization | |
|---|---|---|---|---|
| Stringency | Polynucleotide | | Temperature and | Wash Temperature |
| Condition | Hybrid | Hybrid Length (bp)¹ | Buffer² | and Buffer² |
| A | DNA:DNA | >50 | 65° C.; 1X SSC -or- | 65° C.; 0.3X SSC |
| | | | 42° C.; 1X SSC, 50% | |
| | | | formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, 50% | |
| | | | formamide | |
| D | DNA:RNA | <50 | T_{D}*; 1X SSC | T_{D}*; 1X SSC |
| E | RNA:RNA | >50 | 70° C.; 1X SSC | 70° C.; 0.3xSSC |
| | | | -or- | |
| | | | 50° C.; 1X SSC, 50% | |
| | | | formamide | |
| F | RNA:RNA | <50 | T_{F}*; 1X SSC | T_{f}*; 1X SSC |
| G | DNA:DNA | >50 | 65° C.; 4X SSC | 65° C.; 1X SSC |
| | | | -or- | |
| | | | 42° C.; 4X SSC, 50% | |
| | | | formamide | |
| H | DNA:DNA | <50 | T_{H}*; 4X SSC | T_{H}*; 4X SSC |
| I | DNA:RNA | >50 | 67° C.; 4X SSC | 67° C.; 1X SSC |
| | | | -or- | |
| | | | 45° C.; 4X SSC, 50% | |
| | | | formamide | |
| J | DNA:RNA | <50 | T_{J}*; 4X SSC | T_{J}*; 4X SSC |
| K | RNA:RNA | >50 | 70° C.; 4X SSC | 67° C.; 1X SSC |
| | | | -or- | |
| | | | 50° C.; 4X SSC, 50% | |
| | | | formamide | |
| L | RNA:RNA | <50 | T_{L}*; 2X SSC | T_{L}*; 2X SSC |
| M | DNA:DNA | >50 | 50° C.; 4X SSC | 50° C.; 2X SSC |
| | | | -or- | |
| | | | 40° C.; 6X SSC, 50% | |
| | | | formamide | |
| N | DNA:DNA | <50 | T_{N}*; 6X SSC | T_{N}*; 6X SSC |
| O | DNA:RNA | >50 | 55° C.; 4X SSC | 55° C.; 2X SSC |
| | | | -or- | |
| | | | 42° C.; 6X SSC, 50% | |
| | | | formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, 50% | |
| | | | formamide | |
| R | RNA:RNA | <50 | T_{R}*; 4X SSC | T_{R}*; 4X SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the | | | | |
| # hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀Na⁺) + 0.41 (% G + C) − (600/N), where N is the number of bases in the hybrid, and Na⁺ is the concentration of sodium ions in the hybridization buffer (Na⁺ for 1xSSC = 0.165M). | | | | |

Additional examples of stringency conditions for polynucleotide hybridization are provided in Sambrook et al. (1989) Molecular Cloning: A Laboratory Manual, Chs. 9 & 11, Cold Spring Harbor Laboratory Press, Cold Spring Harbor, NY, and Ausubel et al., eds. (1995) Current Protocols in Molecular Biology, Sects. 2.10 & 6.3-6.4, John Wiley & Sons, Inc., herein incorporated by reference.

The isolated polynucleotides of the present invention may also be used as hybridization probes and primers to identify and isolate DNAs having sequences encoding polypeptides homologous to the disclosed polynucleotides. These homologs are polynucleotides and polypeptides isolated from species different than those of the disclosed polypeptides and polynucleotides, or within the same species, but with significant sequence similarity to the disclosed polynucleotides and polypeptides. Preferably, polynucleotide homologs have at least 60% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

The isolated polynucleotides of the present invention may also be used as hybridization probes and primers to identify cells and tissues that express the polypeptides of the present invention and the conditions under which they are expressed.

The isolated polynucleotides of the present invention may be operably linked to an expression control sequence such as the pMT2 and pED expression vectors for recombinant production of the polypeptides of the present invention. General methods of expressing recombinant proteins are well known in the art.

A number of cell types may act as suitable host cells for recombinant expression of the polypeptides of the present invention. Mammalian host cells include, e.g., COS cells, CHO cells, 293 cells, A431 cells, 3T3 cells, CV-1 cells, HeLa cells, L cells, BHK21 cells, HL-60 cells, U937 cells, HaK cells, Jurkat cells, normal diploid cells, cell strains derived from in vitro culture of primary tissue, and primary explants.

Alternatively, it may be possible to recombinantly produce the polypeptides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of the present invention are made in yeast or bacteria, it may be necessary to modify them by, e.g., phosphorylation or glycosylation of appropriate sites, in order to obtain functionality. Such covalent attachments may be accomplished using well-known chemical or enzymatic methods.

The polypeptides of the present invention may also be recombinantly produced by operably linking the isolated polynucleotides of the present invention to suitable control sequences in one or more insect expression vectors, such as baculovirus vectors, and employing an insect cell expression system. Materials and Methods for baculovirus/Sf9 expression systems are commercially available in kit form (e.g., the MAXBAC® kit, Invitrogen, Carlsbad, Calif.).

Following recombinant expression in the appropriate host cells, the polypeptides of the present invention may then be purified from culture medium or cell extracts using known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the polypeptides of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides of the present invention may also be produced by known conventional chemical synthesis. Methods for chemically synthesizing the polypeptides of the present invention are well known to those skilled in the art. Such chemically synthetic polypeptides may possess biological properties in common with the natural, purified polypeptides, and thus may be employed as biologically active or immunological substitutes for the natural polypeptides.

The polypeptides of the present invention also encompass molecules that are structurally different from the disclosed polypeptides (e.g., which have a slightly altered sequence), but which have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques and kits for such alterations, substitutions, replacements, insertions, or deletions are well known to those skilled in the art.

Antibodies

Antibody molecules capable of specifically binding to the polypeptides of the present invention may be produced by methods well known to those skilled in the art. For example, monoclonal antibodies can be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides of the present invention.

A full-length polypeptide of the present invention may be used as the immunogen, or, alternatively, antigenic peptide fragments of the polypeptides may be used. For example, the immunogen may be a functional motif of the NgR1 (e.g., one or more of the amino acid sequences of SEQ ID NOs:2, 4, 6, 8, 10, 12, 14, and 16) and/or a related peptide or cyclized peptide (e.g., one or more of the amino acid sequences of SEQ ID NOs:18, 20, 22, 24, 26, 27, 28, 29, 30, 31, 32, 33, 34, and 37). An antigenic peptide of a polypeptide of the present invention comprises at least four continuous amino acid residues and encompasses an epitope such that an antibody raised against the peptide forms a specific immune complex with the polypeptide. Preferably, the antigenic peptide comprises at least four amino acid residues, more preferably at least seven amino acid residues, and even more preferably at least nine amino acid residues.

As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide of the present invention to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody display libraries can be found in the literature.

Polyclonal sera and antibodies may be produced by immunizing a suitable subject with a polypeptide of the present invention. The antibody titer in the immunized subject may be monitored over time by standard techniques, such as with ELISA using immobilized marker protein. If desired, the antibody molecules directed against a polypeptide of the present invention may be isolated from the subject or culture media and further purified by well known techniques, such as protein A chromatography, to obtain an IgG fraction.

Fragments of antibodies to the polypeptides of the present invention may be produced by cleavage of the antibodies in accordance with methods well known in the art. For example, immunologically active F(ab′) and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques. Humanized antibodies may also be produced using transgenic mice that are incapable of expressing endogenous immunoglobulin heavy and light chain genes, but that can express human heavy and light chain genes.

In some embodiments, the invention provides single domain antibodies. Single domain antibodies can include antibodies whose CDRs are part of a single domain polypeptide. Examples include, but are not limited to, heavy chain antibodies, antibodies naturally devoid of light chains, single domain antibodies derived from conventional four-chain antibodies, engineered antibodies and single domain scaffolds other than those derived from antibodies. Single domain antibodies may be any of those known in the art, or any future single domain antibodies. Single domain antibodies may be derived from any species including, but not limited to, mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in, e.g., WO 94/04678. This variable domain derived from a heavy chain antibody naturally devoid of light chain is known herein as a VHH or nanobody, to distinguish it from the conventional VH of four-chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHH molecules are within the scope of the invention.

In addition to antibodies for use in the instant invention, other molecules may also be employed to modulate the activity of polypeptides of the present invention. Such molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Screening Assays and Sources of Test Compounds

The polynucleotides and polypeptides of the present invention may also be used in screening assays to identify pharmacological agents or lead compounds for other antagonists to NgR1 ligands, which may be used to antagonize (e.g., reverse, decrease, reduce, prevent, etc.) NgR1L-mediated inhibition of axonal growth. For example, samples containing an antagonist of the invention, e.g., a polypeptide comprising an amino acid sequence selected from the group consisting of SEQ ID NOs:2, 4, 6, 10, 14, 18, 22, and 26-34, and an NgR1 ligand (including an NgR1 binding fragment of an NgR1 ligand (e.g., NEP1-40)) can be contacted with one of a plurality of test compounds (e.g., small organic molecules or biological agents), and the interaction in each of the treated samples can be compared to the interaction of the antagonist of the invention and an NgR1 ligand in untreated samples or in samples contacted with different test compounds to determine whether any of the test compounds provides a substantially decreased level of antagonist:NgR1 ligand interactions. In a preferred embodiment, the identification of test compounds capable of modulating the activity of antagonist:NgR1 ligand interactions is performed using high-throughput screening assays, such as provided by BIACORE® (Biacore International AB, Uppsala, Sweden), BRET (bioluminescence resonance energy transfer), and FRET (fluorescence resonance energy transfer) assays, as well as ELISA. One of skill in the art will recognize that test compounds capable of decreasing levels of antagonist:NgR1 ligand interactions may be antagonists of NgR1L (e.g., because they bind to NgR1L and block NgR1:NgR1L interactions) or agonists of NgR1L (e.g., because they bind to, e.g., KFRG and activate inhibition of axonal growth). Such antagonistic or agonistic test compounds screened in the above-described manner may then be further distinguished, e.g., tested for their ability to antagonize NgR1L-mediated axonal growth inhibition, or to enhance NgR1L-mediated axonal growth inhibition, respectively, using well-known methods, e.g., the neurite outgrowth assay described in Example 1.1.

The test compounds of the present invention may be obtained from a number of sources. For example, combinatorial libraries of molecules are available for screening. Using such libraries, thousands of molecules can be screened for inhibitory activity. Preparation and screening of compounds can be screened as described above or by other methods well known to those of skill in the art. The compounds thus identified can serve as conventional “lead compounds” or can be used as the actual therapeutics.

Methods of Treatment

Peptide mimetics related to functional motifs of the NgR1, particularly peptides comprising the amino acid sequence of KFRG, may be used as antagonists to the axonal growth inhibition effects of NgR1 ligands, e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1. As such, the present invention provides both prophylactic and therapeutic methods for treatments requiring axonal regeneration, i.e., antagonism (e.g., reversal, decrease, reduction, prevention, etc.) of axonal growth inhibition, that involve administration of an antagonist of the invention. A skilled artisan will recognize that such methods of treatment will be particularly useful in subjects who may suffer from, or who suffer from, or who may have suffered from, a brain injury caused by, e.g., stroke, multiple sclerosis, Parkinson's disease, Alzheimer's disease, etc. The methods involve contacting cells (either in vitro, in vivo, or ex vivo) with an antagonist of the invention in an amount effective to antagonize (e.g., reverse, decrease, reduce, prevent, etc.) the activity of NgR1 ligands, e.g., the biological consequences of one or more NgR1 ligands binding to the NgR1 complex in neurons (e.g., the inhibition of axonal growth and/or the formation of the higher order receptor-signaling complex). The antagonist may be any molecule that antagonizes the activity of NgR1 ligands, including, but not limited to, small molecules and peptide inhibitors.

For example, small molecules (usually organic small molecules) that antagonize the activity of NgR1 ligands (e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1) may be used to, e.g., reverse NgR1 ligand-mediated axonal growth inhibition. Novel antagonistic small molecules may be identified by the screening methods described above, and may be used in the treatment methods of the present invention described here.

Decreased activity of NgR1 ligands in an organism in need of axonal regeneration but afflicted with (or at risk for) inhibition of axonal growth mediated by NgR1 ligands, or in an involved cell from such an organism, may also be achieved using peptide inhibitors, e.g., the mimetic peptide antagonists of the invention, that bind to and inhibit the activity of NgR1 ligands. Peptide inhibitors include peptide pseudosubstrates that prevent NgR1 ligands from interacting with the NgR1. Peptide inhibitors that antagonize, or may antagonize, NgR1 ligands are disclosed herein as mimetic peptide antagonists, and include, but are not limited to, KFRG (SEQ ID NO:26), LQKFRGSS (SEQ ID NOs:14 and 16), KFRGS (SEQ ID NOs:18 and 20), and QKFRG (SEQ ID NO:22 and 24). In some embodiments, these peptide inhibitors are cyclized via disulfide bonds (e.g., SEQ ID NOs:31, 32, 33, and 34) to improve the ability of the peptides to act as antagonists (see Williams et al. (2000) J. Biol. Chem. 275(6):4007-12; Williams et al. (2000a) Mol. Cell. Neurosci. 15(5):456-64). Cyclized and noncyclized NgR1 ligand peptide inhibitors may be chemically synthesized. Additionally, the peptide inhibitors of the invention may be acetylated and/or amide blocked using well-known methods. One can provide a cell (e.g., a neuron) with a peptide inhibitor in vitro, in vivo, or ex vivo using the techniques described below.

The NgR1 is an important target for methods of treatment of, e.g., neurodegenerative disorders, at least because it is a key ligand-binding molecule in a higher-order receptor complex that mediates inhibitory signaling for at least three myelin molecules. If this complex limits regeneration in the damaged brain, then agents that interfere with ligand binding would have therapeutic potential. Until recently, no known small binding motifs had been identified in the NgR1. However, LRR-motif proteins might use an evolutionarily conserved mechanism to engage ligands, and functional motifs in one receptor might be deduced from the identification of functional motifs in a second receptor. Testing of peptide mimetics of four NgR1 exposed loops was conducted to research their ability to antagonize the inhibitory activity of MAG, one of the key myelin ligands for the NgR1. All of the peptides were constrained by a disulfide bond, as this procedure often increases the efficacy of “loop” peptide mimetics by constraining them in a configuration that shares structural overlap with the sequence in the native protein structure (Hruby (2002) Nat. Rev. Drug Discov. 1(11):847-58; Williams et al. (2000) supra). Three of the peptides had little or no activity; however, it remains possible that these sequences do harbor functional motifs that have been constrained in an inappropriate manner and/or are important for the function of other NgR1 ligands. One of the peptides, NRL2, was an effective MAG antagonist, with near maximal inhibitory activity seen at ˜50 μg/ml (˜45 μM).

Gangliosides, and in particular GT1b and GD1a, are candidate coreceptors for MAG in neurons. In this context, a considerable body of evidence supports the view that GT1b is a neuronal receptor for MAG (Venkatesh et al. (2005) supra; Collins et al. (1997) supra; Fujitani et al. (2005) supra; Vinson et al. (2001) supra) and antibodies to GT1b can immunoprecipitate p75NTR (Fujitani et al. (2005) supra; Yamashita et al. (2002) supra) and presumably other members on the NgR1 complex. Several investigators have also demonstrated that antibodies that cluster GT1b can fully mimic MAG inhibition of neurite outgrowth (Fujitani et al. (2005) supra; Vinson et al. (2001) supra; Vyas et al. (2002) supra; Williams et al. (2005) supra; Lehmann et al. (2007) J. Neurosci. 27(1):27-34); one explanation is that they do so by coclustering the p75NTR/NgR1/Lingo complex.

In the present studies, the inventors investigated how gangliosides might interact with the NgR complex, guided by studies on ganglioside interactions with MAG itself. In this context, arginine 118 is part of an FRG motif in MAG that recognizes terminal sialic acid residues on gangliosides and perhaps other glycoconjugates (Vinson et al. (2001) supra; Tang et al (1997a) supra); this fact raised the question as to whether it is simply a coincidence that the NgR family also contains up to three conserved FRG motifs.

Using sedimentation assays, evidence was obtained that GT1b can interact with the NgR, albeit at low μM concentrations. At these concentrations, GT1b forms micelles that migrate with a sedimentation coefficient of ca. 4.5, corresponding to approximately 10-12 molecules per micelle (Formisano et al. (1979) Biochemistry 18:1119-24). This would apparently account for the relatively large shift in the sedimentation coefficient of the NgR1 that is induced, in a dose-dependent manner, by GT1b. The binding appeared to be sialic acid-dependent, as the same shift can be induced by the much simpler GM1 ganglioside that shares a common terminal sialic acid with GT1b. The demonstration that asialo-GM1 does not induce a shift is consistent with the binding being mediated directly by the terminal sialic acid. It is worth noting that gangliosides are present in neuronal membranes at high concentrations (Wang et al. (1998) Compar. Biochem. Physiol. 199:435-39), and that productive interactions with neuronal receptors in the same membrane need not involve high-affinity interactions.

Several lines of evidence speak to the specificity of the GT1b/NgR interaction. In this context, asialo-GM1 did not interact with the NgR1, and this is expected for an interaction predicted to be mediated by the terminal sialic acid. However, individual single point mutations within the three independent FRG motifs in the NgR1, mutations that would have no appreciable effect on the structure or overall surface charge of the NgR1, each substantially reduced the interaction. In each case, R to E mutations within the motifs resulted in less binding between GT1b and the mutated receptors determined at high (i.e., close to saturation) concentrations of GT1b. This suggests that GT1b can interact with at least three spatially distinct sites on the NgR1. Whereas complex formation was reduced by ˜50% when R151 or R279 were mutated, it was reduced by ˜70% when R199 was mutated, suggesting that the latter site might play a greater role in sialic acid binding. The protein structure of the NgR1 extracellular domain was examined using a number of computational approaches to identify potential small molecule binding sites or pockets on the surface; interestingly all FRG-containing sites formed part of larger potential binding pockets in either the side or convex surface of the protein (data not shown). Of the three NgR1FRG motifs, two are conserved in NgR2 (R151 and R199) and two are conserved in NgR3 (R199 and R279). The conservation of the site around R199 in all three receptors might argue for a more important function for this motif, and indeed mutation at this site had the most dramatic effect on GT1b binding. R199 also has three neighboring arginines (196, 223, and 175) arranged in a cluster that may play a key role in forming the site of a binding pocket for GT1b and/or another sialic acid-containing glycoconjugate.

The data implicate NgR1FRG motifs as candidate binding sites for the sialic acid moiety on gangliosides and perhaps other glycoconjugates. However, some residual GT1b/NgR1 complex formation (˜30%) could still be seen after mutating R199, with a similar level seen following mutation of all three of the arginines in all three FRG motifs (data not shown). This suggests that the residual GT1b binding might involve additional sites. Nonetheless, it is also possible that residual binding might reflect a lower affinity interaction with one or more of the mutated FRG sites (based on studies on MAG itself in which mutation of arginine 118 (within an FRG motif) has been interpreted as reducing the affinity, rather than abolishing the binding, of sialic acid to the site (Vinson et al. (2001) supra)).

An independent way to test if FRG motifs are important for ganglioside function is to test if FRG peptides can function as ganglioside antagonists. Inhibition of a biological response with a small peptide is usually more sensitive (and more pertinent) than inhibition of a direct binding response due to the nonphysiological nature of binding assays. Of the three FRG motifs present in the NgR1, one is contained in an exposed amino-terminal loop that lends itself well to a strategy for making a cyclic peptide mimetic of the loop. In this context, constraining a loop sequence by a disulphide bond often holds the mimetic in a configuration that shares structural overlap with the sequence in the native protein structure. In this context, a constrained cyclic peptide mimetic of the FRG-containing NgR1 loop sequence did in fact function as a full GT1b antagonist in that it fully prevented the inhibition of neurite outgrowth normally seen following antibody-induced clustering of GT1b in neurons. Therefore, two direct independent lines of investigation support the hypothesis that GT1b can interact with the NgR1, and perhaps other NgR5, by interacting with FRG motifs.

Under some circumstances, GT1b appears to be able to serve as a coreceptor for MAG, presumably by increasing MAG's affinity and/or interaction with the NgR complex. If this depends upon the aforementioned GT1b/NgR interaction, one prediction is that a peptide that inhibits GT1b function should also inhibit soluble MAG function. In the present studies, the NRL2 peptide was an effective soluble MAG antagonist, with near maximal inhibitory activity seen at ˜50 μg/ml (˜45 μM). Moreover, in control studies, the inventors demonstrated that peptide mimetics of the other three exposed loops on the NgR do not function as soluble MAG antagonists.

The use of short peptides distilled the inhibitory activity of the NRL2 peptide down to a four amino acid motif (KFRG). Alanine substitutions within this motif showed that the first amino acid could be substituted without any obvious effect on peptide function. In contrast, substitution of the phenylalanine resulted in a ˜2-fold reduction in peptide activity, with substitution of the arginine or glycine resulting in a complete loss of activity when tested at up to 100 μg/ml. This demonstrates that the FRG triplet is the minimal functional motif within the peptide.

In order to directly test whether the FRG motif plays a role in MAG binding to the NgR1, MAG binding to mutated full-length receptors expressed in cells was measured. Mutations of the two highly solvent-exposed charged residues within the KFRG motif in the full-length NgR1 to either aspartic acids or alanines reduced the binding of MAG by ˜60%. Single alanine substitution experiments demonstrated that arginine 279 is more important for MAG binding than lysine 277. Therefore, based on two independent lines of evidence (peptide competition and site-directed mutagenesis), the KFRG motif in the terminal loop region of the NgR1 has been identified as a site that can play a role in MAG binding. As seen in control experiments, the mutations have no obvious effect on the interaction between the NgR1 with itself, or with p75^{NTR}. Also, the same mutations had no significant effect on the binding of soluble Nogo-66-AP to the receptor (data not shown).

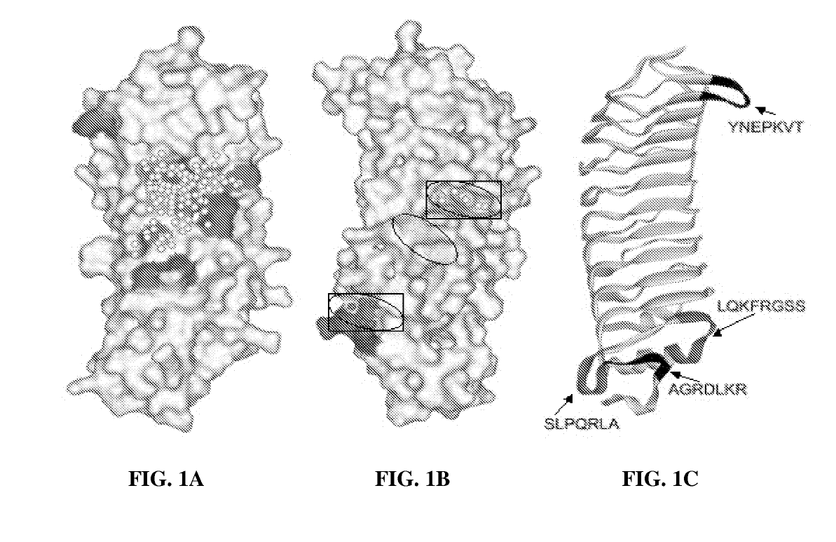

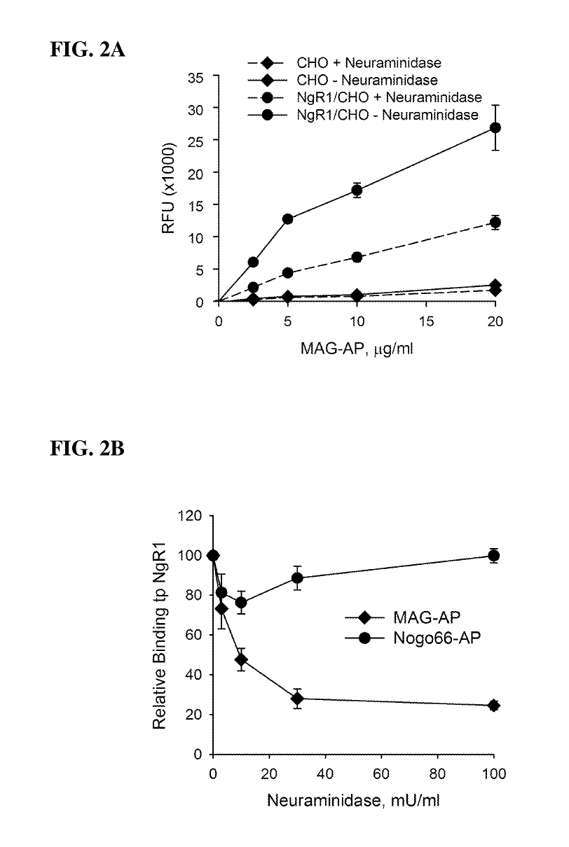

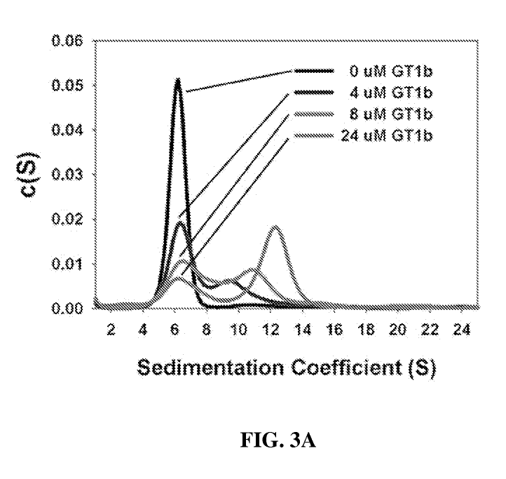

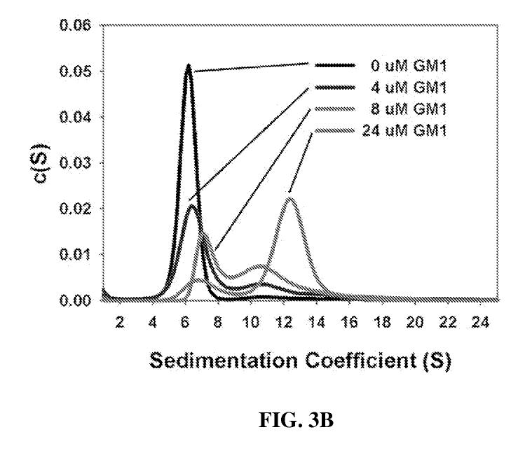

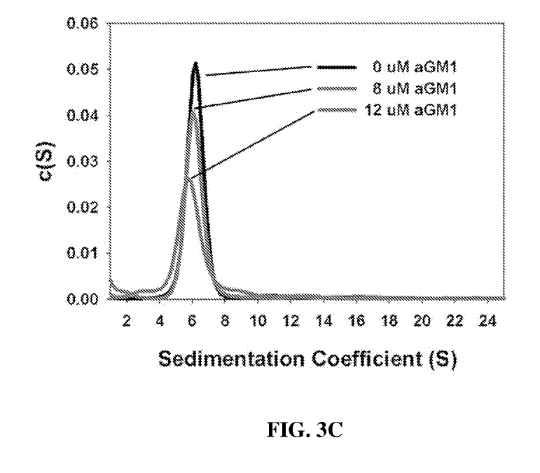

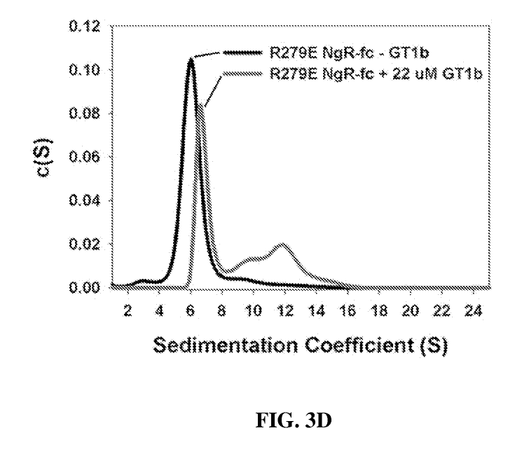

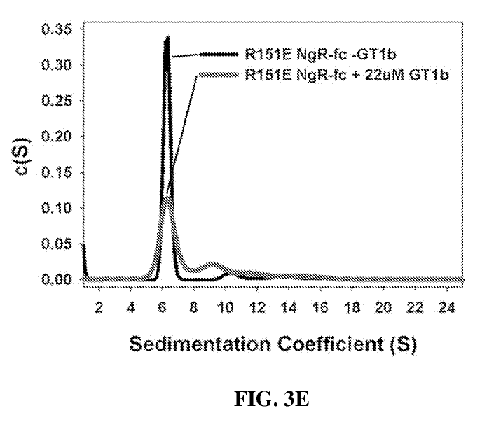

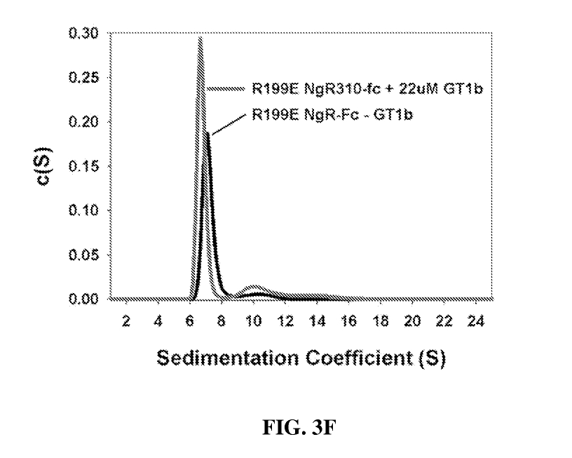

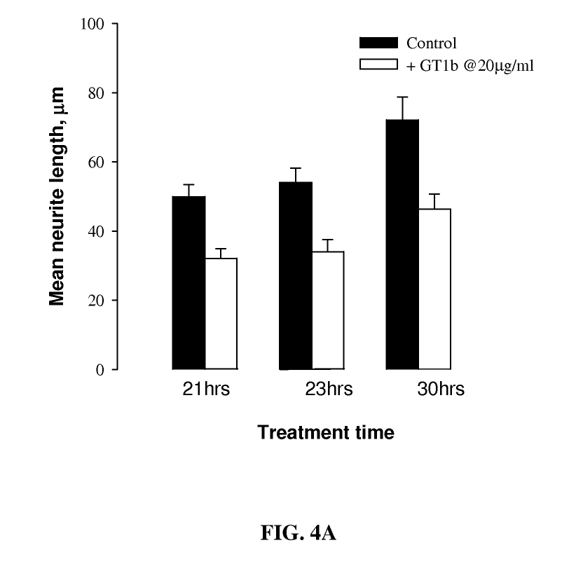

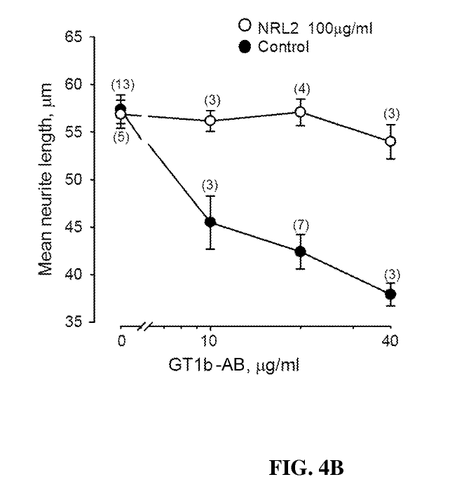

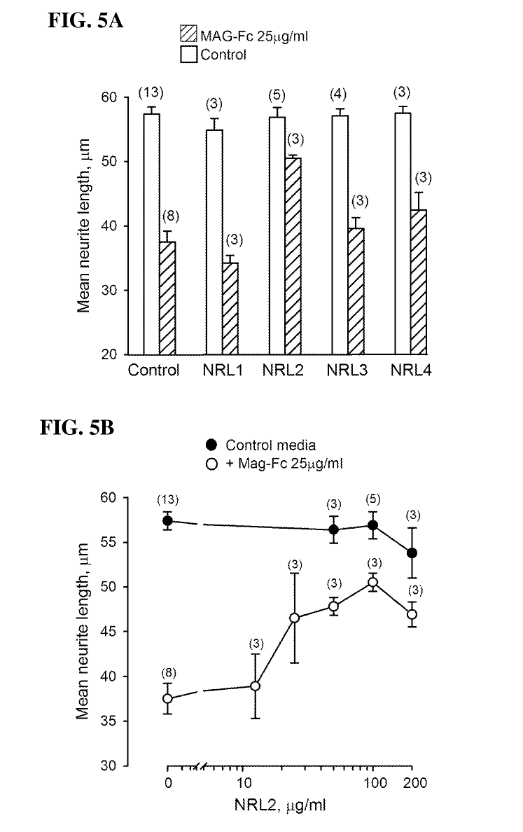

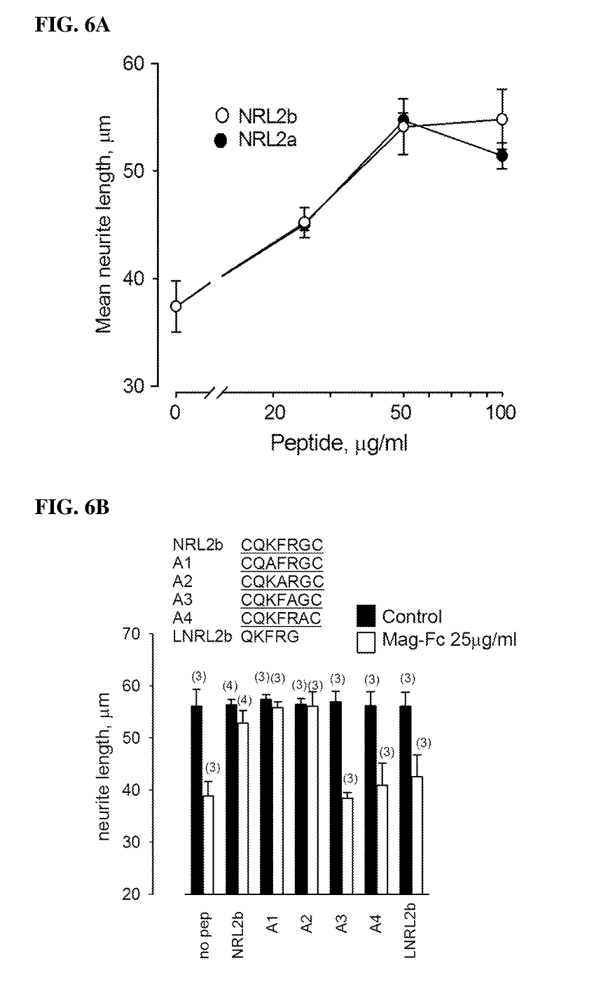

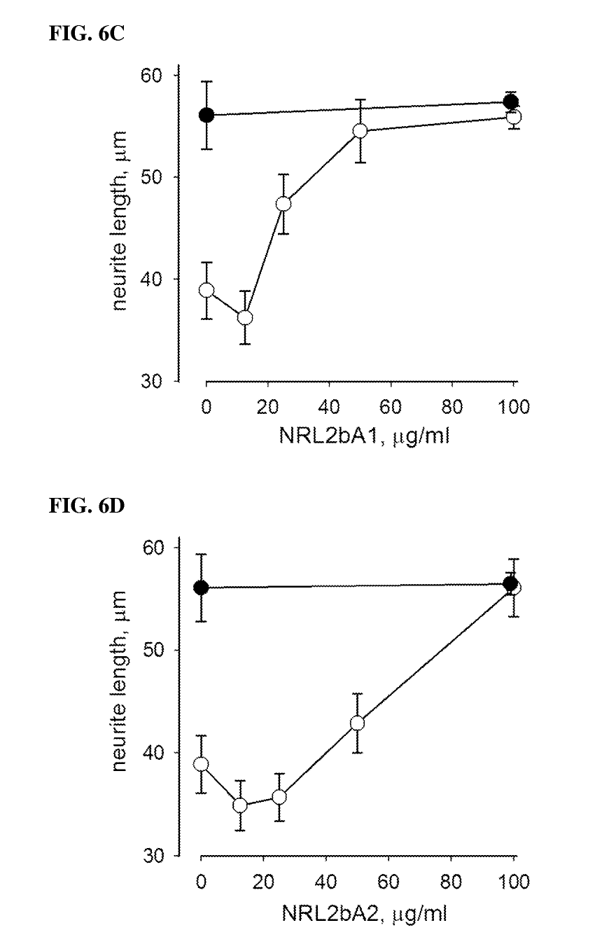

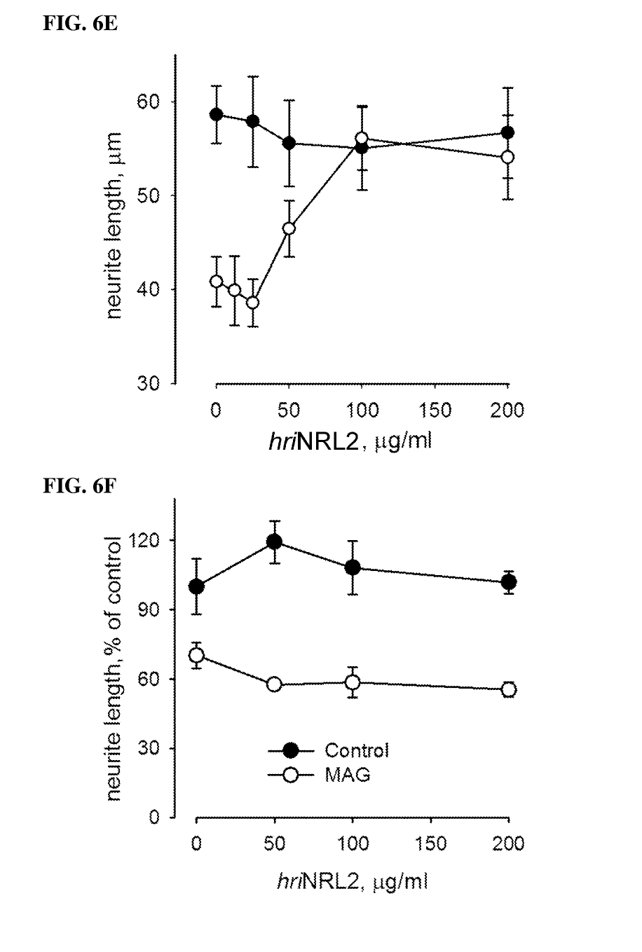

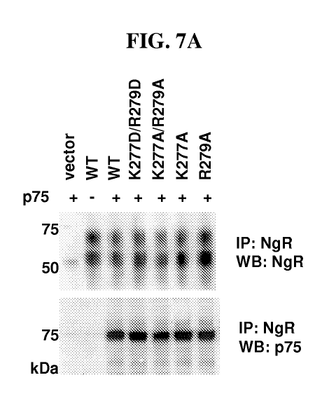

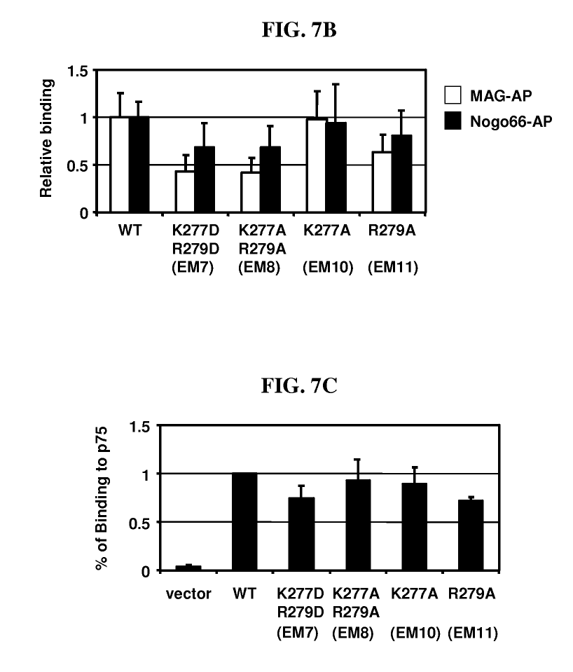

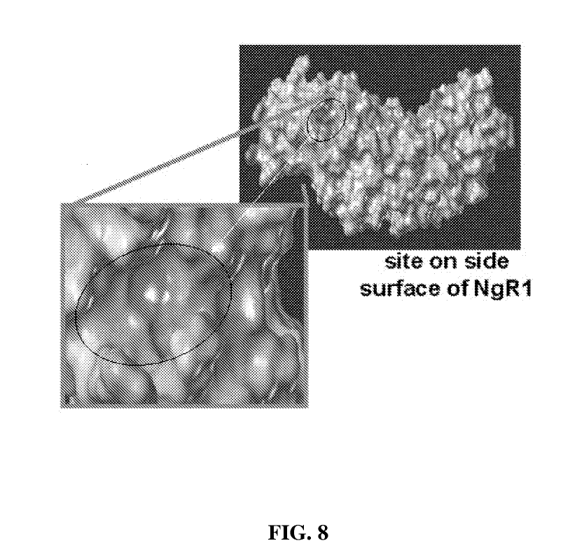

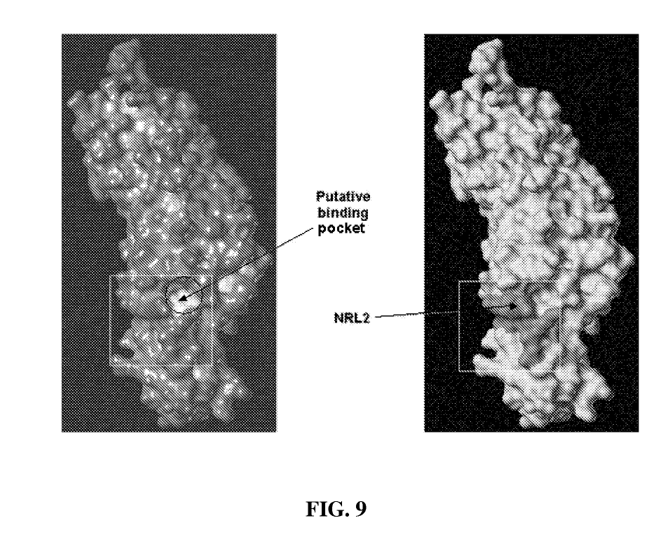

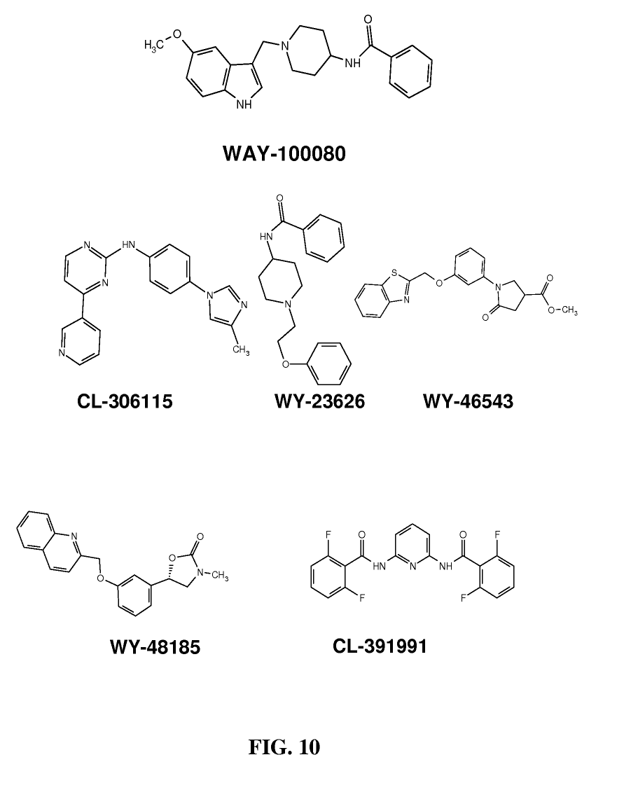

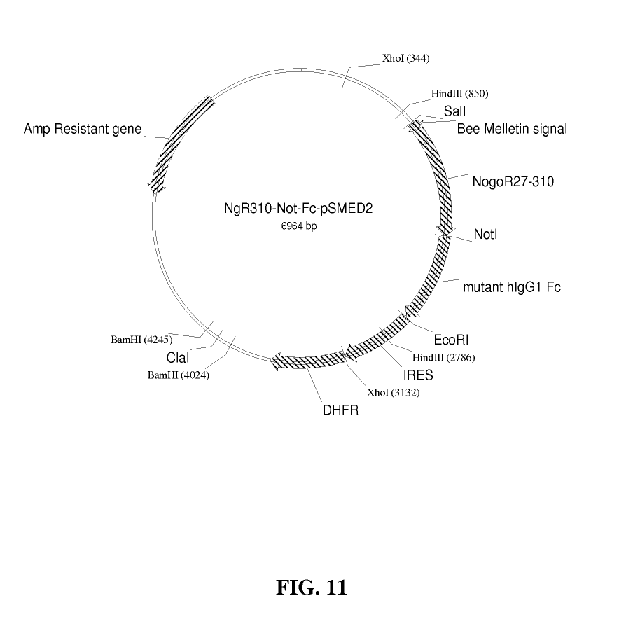

A number of lines of evidence support the hypothesis that the FRG motif is likely to play an indirect role in MAG binding. First, mutations within the site reduced rather than completely inhibited MAG binding. Second, an extensive mutagenesis study has mapped the MAG binding site to a different region of the receptor (see FIG. 1). Third, a computational approach suggests that this site of the NgR is more likely to interact with a small ligand as opposed to a protein ligand. Finally, as demonstrated in a direct binding assay, mutations within the same FRG motif attenuate the binding of GT1b, an established coreceptor for MAG, to the NgR1.

One conclusion from the results of this study is that, in addition to serving as sialic acid-binding sites on MAG itself, FRG motifs within the NgR are also binding sites for the terminal sialic acid moiety on gangliosides, and perhaps other glycoconjugates. One possibility is that GT1b might facilitate soluble MAG binding to the NgR by cross-linking both molecules via their shared FRG motifs. The inventors have shown that NgR-derived FRG motif peptides can inhibit the function of soluble MAG. However, MAG apparently has an additional “inhibitory” binding site that can most probably interact directly with the NgR and can, in some circumstances, act independently of the sialic acid-binding site. Likewise, the other myelin inhibitors bind to the NgR at sites that are distant from the FRG motifs. This probably accounts for the failure of the FRG peptides to overcome the inhibitory activity of substrate-bound MAG and myelin. Thus, the FRG peptides are unlikely to offer therapeutic opportunities in circumstances where myelin is inhibiting regeneration. However, a recent study showed that passive immunization with anti-ganglioside antibodies directly inhibits axonal regeneration after axonal injury in mice (Lehmann et al. (2007) supra). A considerable body of evidence also exists suggesting that autoimmune, anti-ganglioside antibodies might contribute to the poor prognosis of some patients with peripheral neuropathies (Willison and Yuki (2002) Brain 125(Pt. 12):2591-625). The results obtained in the present study might be of value in considering therapeutic opportunities for peripheral neuropathies in which antibodies to gangliosides might play a pathologic role.

In this study, the NgR1-derived NRL2 peptide fully inhibited the response induced by the GT1b antibody, suggesting that it can interfere with the interaction between GT1b and the NgR1 complex. The evidence that GT1b can bind, albeit with low affinity, to highly conserved FRG motifs in the NgR1 supports this model. However, in the absence of additional evidence for direct GT1b binding to the NgR1, it remains possible that the peptides perturb an additional and/or alternative GT1b interaction. Nonetheless, the fact that the NgR1 NRL2 peptide inhibits the response to soluble MAG and the GT1b antibody conforms with the concept that soluble MAG functions by clustering a GT1b/NgR complex in neurons. In this context, it is well established that antibody-induced clustering of GT1b in neurons fully mimics the inhibitory activity of MAG (Vinson et al. (2001) supra; Vyas et al. (2002) supra; Williams et al. (2005) supra). This supports the hypothesis that GT1b can be an integral component of the functional receptor complex for MAG in neurons, and extends it by suggesting that GT1b can play a role in stabilizing MAG binding to the NgR1 via simultaneous engagement of shared FRG motifs.

Administration

Any of the compounds described herein (preferably a mimetic peptide or small molecule antagonist of the invention) can be administered in vivo in the form of a pharmaceutical composition for treatments requiring antagonism of axonal growth inhibition, i.e., axonal regeneration. The pharmaceutical composition may be administered by any number of routes, including, but not limited to, oral, nasal, intraventricular, rectal, topical, sublingual, subcutaneous, intravenous, intramuscular, intraarterial, intramedullary, intrathecal, intraperitoneal, intraarticular, or transdermal routes. In addition to the active ingredients, the pharmaceutical composition(s) may contain a pharmaceutically acceptable carrier(s). Such compositions may contain, in addition to any of the compounds described herein and an acceptable carrier(s), various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

For any compound, the therapeutically effective dose can be estimated initially either in cell culture or in animal models. The therapeutically effective dose refers to the amount of active ingredient that ameliorates the condition or its symptoms. Therapeutic efficacy and toxicity in cell cultures or animal models may be determined by standard pharmaceutical procedures (e.g., ED₅₀: the dose therapeutically effective in 50% of the population; LD₅₀: the dose lethal to 50% of the population). The dose ratio between therapeutic and toxic effects is the therapeutic index, and can be expressed as the ratio ED₅₀/LD₅₀. Pharmaceutical compositions that exhibit large therapeutic indexes are preferred.

The data obtained from cell culture and animal models can then be used to formulate a range of dosages for the compound for use in mammals, preferably humans. The dosage of such a compound preferably lies within a range of concentrations that includes the ED₅₀ with little to no toxicity. The dosage may vary within this range depending upon the composition form employed and the administration route utilized.

Another aspect of the present invention relates to kits for carrying out the administration of NgR1 ligand antagonists (e.g., the peptide mimetic antagonists of the invention), either alone or with another therapeutic compound(s) or agent(s). In one embodiment, the kit comprises one or more NgR1 ligand antagonists formulated with a pharmaceutically acceptable carrier(s).

The entire contents of all references, patents, and patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

### Example 1

### Materials and Methods

### Example 1.1

### Neurite Outgrowth Assays

Cerebellar neurons isolated from postnatal day ⅔ rat pups were cultured over monolayers of 3T3 cells (Doherty et al. (1991) Neuron 6(2):247-58) essentially as previously described (Williams et al. (1994) Neuron 13(3):583-94). Monolayers were established by seeding ˜80,000 cells into individual chambers of an eight-chamber tissue culture slide coated with poly-L-lysine and fibronectin. The cell lines, and monolayers, were maintained in Dulbecco's modified Eagle's medium supplemented with 10% fetal calf serum (FCS). Cocultures were established by removing the media from the monolayers and seeding ˜6000 dissociated cerebellar neurons into each well in SATO medium (modified from Doherty et al. (1990) Neuron 5(2):209-19; Dulbecco's modified Eagle's medium supplemented with 2% FBS, 33% bovine albumin, 0.62 μg/ml progesterone, 161 μg/ml putrescine, 4 μg/ml L-thyroxine, 0.387 μg/ml selenium, and 3.37 μg/ml tri-iodo-thyronine (components from Sigma-Aldrich, St. Louis, Mo.)). Monolayers were established for 24 hours prior to addition of the neurons and the cultures were maintained for ˜23-27 hr. Following careful fixation with 4% paraformaldehyde, the neurons were stained with a GAP-43 antibody, and the mean length of the longest neurite per cell was measured for ˜120-150 neurons, again as previously described (Williams et al. (1994) supra). For neurite outgrowth on substrate-bound MAG, 96-well plates were coated with a thin layer of nitrocellulose (Bio-Rad, Hercules, Calif.) before incubating with 1 μg/ml of MAG(d1-5) (a chimeric construct containing domains 1-5 of the extracellular portion of MAG) at 4° C. overnight. Wells were subsequently coated with 17 μg/ml of poly-D-lysine (Sigma, St. Louis, Mo.), followed by incubation in Dulbecco's modified Eagle's medium containing 10% FCS. Cerebellar granule neurons were dissociated and seeded at a density of 10⁴ cells per well. Cells were cultured for 18-20 h before being fixed with 4% paraformaldehyde and stained with a neuronal-specific anti-III-tubulin antibody, Tuj 1 (Covance, Emeryville, Calif.). The average of total neurite lengths from each neuron was measured automatically by the MetaXpress Neurite Outgrowth module (Molecular Devices, Sunnyvale, Calif.) from at least 200 neurons per well, in triplicate wells per experiment. Results were repeated independently more than three times.

### Example 1.2

### Structures

For the purposes of molecular modeling, the 1M10 (pdb accession number) glycoprotein Ib alpha in complex with von Willebrand factor (Huizing a et al. (2002) Science 297:1176-79) and the 1OZN (pdb accession number) structure of the NgR1 (He et al. (2003) Neuron 38(2):177-85) were used. Swiss PDB software packages were used to isolate the structure of various motifs from the binding interfaces of the crystals, and Accelrys software was used to generate images.

### Example 1.3

### Reagents

Synthetic peptides were all obtained from a commercial supplier (Multiple Peptide Systems, San Diego, Calif.). All peptides were purified to the highest grade by reverse-phase HPLC and obtained at the highest level of purity (>97%). With all peptides, there was no indication of higher molecular weight species. Where peptide sequences are underlined, this denotes a peptide that has been cyclized via a disulfide bond between the given cysteine residues. All peptides were acetylated (e.g., denoted with “N-Ac-”) and amide blocked (e.g., denoted with “—NH₂”). Recombinant MAG-Fc chimera was obtained from R&D Systems (Minneapolis, Minn.) and used at final concentrations ranging from 5-25 μg/ml. The monoclonal antibody to GT1b (clone GMR5) was obtained from Seikagaku America (Falmouth, Mass.) and was used at a final concentration of 20 μg/ml. All reagents were diluted into the coculture media and, in general, added to the cultures just prior to the plating of the neurons. GT1b and GM1 were obtained as gifts from Dr. Gino Toffano (Libero, Italy) and University of Milan, and asialo-GM1 was obtained from Sigma (St. Louis, Mo.). The recombinant NgR1(310)-fc and MAG(d1-5) chimeras were expressed and purified in-house. Pharmacological reagents were obtained from Calbiochem (La Jolla, Calif.) and/or Sigma. For reagents used in cell-surface NgR binding assays, please see Examples 1.5 and 1.7. For reagents used in the cell surface p75NTR-NgR-AP binding assay, please see Example 1.13.

### Example 1.4

### Construction of Nogo Receptor 1 Mutants

Human Nogo Receptor 1 (NgR1) point mutants (EM7 (227D/R279D); EM8 (K277A, R279A); EM10 (K277A) and EM11 (R279A)) were constructed using the Quikchange XL site-directed mutagenesis kit (Stratagene) following the manufacturer's recommended protocol. The wild-type human NgR1 cDNA (IMAGE:2121045 3 (SEQ ID NO:61); corresponding to GENBANK Accession No. NM_{—}023004 (SEQ ID NO:62) and Gene ID No. 65078) in a mammalian expression vector was used as a template to construct all the described mutants. Mutagenic oligonucleotide sequences used were as follows in Table 2:

TABLE 2

| | | SEQ |
|---|---|---|
| | | ID |
| Mutant | Primer sequences from 5′ to 3′ | NO: |
| EM7 | CTGGGCCTGGCTGCAGGACTTCGATGGCTCCTCCTCCGAG | 38 |
| | CTCGGAGGAGGAGCCATCGAAGTCCTGCAGCCAGGCCCAG | 39 |
| EM8 | CTCTGGGCCTGGCTGCAGGCGTTCGCCGGCTCCTCCTCCGA | 40 |
| | GGTGCCCTGC | |
| | GCAGGGCACCTCGGAGGAGGAGCCGGCGAACGCCTGCAGCC | 41 |
| | AGGCCCAGAG | |
| EM10 | CTCTGGGCCTGGCTGCAGGCGTTCCGCGGCTCCTCCTCCG | 42 |
| | CGGAGGAGGAGCCGCGGAACGCCTGCAGCCAGGCCCAGAG | 43 |
| EM11 | GCCTGGCTGCAGAAGTTCGCCGGCTCCTCCTCCGAGGTGC | 44 |
| | GCACCTCGGAGGAGGAGCCGGCGAACTTCTGCAGCCAGGC | 45 |

### Example 1.5

### Cell-surface NgR Binding Assay

COS-7 cells were cotransfected with either wild type or mutant NgR1 constructs along with a CMV-beta-galactosidase plasmid (pCMVb, BD Biosciences, San Jose, Calif.) as a transfection control. Transfection was performed in 6-well plates using Lipofectamine 2000 (Invitrogen, Carlsbad, Calif.) following the manufacturer's protocol. The next day, cells were trypsinized and seeded at 30,000 cells per well in duplicate polylysine-coated 96-well plates (BD Biosciences); one plate was used in the binding assay, and the other was used to correct for transfection efficiency by measuring beta-galactosidase activity (described below). The remaining cells were separately plated and assayed for surface expression of the NgR1 proteins by immunocytochemistry (Example 1.9) and for total NgR1 protein levels by Western blot analysis (Example 1.8). All mutant proteins were expressed on the cell surface and produced in comparable amounts to the wild type protein (data not shown). The next day, wells were rinsed once with HBAH (Hank's Balanced Salt Solution (HBSS) containing bovine serum albumin (0.5 mg/ml), NaN₃ (0.1%), and 20 mM HEPES pH 7.0) at room temperature followed by incubation with 100 μl of AP fusion protein (MAG-AP or Nogo-66-AP) diluted to a final concentration of 10 μg/ml in HBAH for 90 minutes. Wells were then washed six times with gentle shaking in HBAH at room temperature, five minutes each wash. Cells were then fixed with acetone-formaldehyde (60%-3%, in 20 mM HEPES, pH 7.0) for 15 seconds at room temperature, then washed three times for five minutes each with HBSS. Binding of AP-tagged ligands was measured using the Great EscAPe SEAP Kit (BD Biosciences) following the manufacturer's recommended protocol. Briefly, after aspirating HBSS, 60 μl of dilution buffer was added to each well, the plates were sealed, and then incubated at 65° C. for 90 min. Plates were cooled on ice and then 60 μl of assay buffer was added per well and incubated at room temperature for five minutes. Sixty microliters of diluted CSPD® substrate (Applied Biosystems, Foster City, Calif.) was then added per well, incubated for 10 minutes at room temperature, and then read on an LMAXII luminometer (Molecular Devices). Absolute binding numbers were corrected by subtracting average binding values obtained from mock-transfected controls. Binding was further corrected for sample-to-sample variations in transfection efficiency by normalizing to beta-galactosidase activity. Beta-galactosidase activity was measured using the luminescent beta-gal detection kit II (BD Biosciences) following the manufacturer's recommended protocol. Three independent binding experiments were conducted with six to eleven replicates per experiment. Background-subtracted, beta-galactosidase-corrected binding values were expressed relative to the wild type receptor.

### Example 1.6

### Statistical Analysis of Cell-surface NgR Binding Assay

Separate statistical analysis was performed for MAG-AP and Nogo-66-AP binding; a linear mixed model was fitted to the data using the receptors as the fixed effects and the experiments, replicates within each experiment, and receptor×experiment interactions as the random effects. The replicates were modeled as random effects for two reasons: information on replicate order was not available, and different numbers of replicates were used in different experiments. Pairwise comparisons were performed between receptors in the framework of the linear mixed model, and raw p-values and Tukey-Kramer multiplicity-adjusted p-values were calculated. The corresponding 95% confidence intervals were also calculated.

### Example 1.7

### Preparation of AP-tagged Fusion Proteins

A fusion protein containing an N-terminal human placental alkaline phosphatase (AP) and a C-terminal Nogo-66 domain was constructed (see, e.g., U.S. Patent Application No. 60/703,134, filed Jul. 28, 2005, hereby incorporated by reference herein it its entirety). Briefly, nucleotide sequences encoding amino acids 1055-1120 of human NogoA (reticulon-4, NP_{—}065393) were ligated to sequences encoding amino acids 23-511 of AP(NM_{—}001632). This fusion was further modified by changing amino acid 47 of the Nogo-66 sequence from cysteine to valine and introducing six consecutive histidine residues at the C-terminus (referred to as Nogo-66-AP(C47V)). The C47V amino acid substitution was introduced using the Quikchange XL site-directed mutagenesis kit (Stratagene) according to the manufacturer's recommended protocol with the following oligonucleotides:

(SEQ ID NO:50)

5′-CTGCTCTTGGTCATGTGAACGTAACGATAAAG
GAGCTCAGGCG-3′

(SEQ ID NO:51)

5′-CGCCTGAGCTCCTTTATCGTTACGTTCACATGAC
CAAGAGCAG-3′.

(SEQ ID NO:51). The coding sequence was inserted into a mammalian expression vector and transiently transfected into HE 293GT cells (Invitrogen) using Lipofectamine 2000 (Invitrogen). The next day, serum-free medium (Free Style 293, Invitrogen) was added and cells were incubated for 48 hours prior to collection of crude conditioned medium. Nogo-66-AP(C47V) concentration was determined by measuring alkaline phosphatase activity and by Western blot analysis for alkaline phosphatase. A stable CHO cell line expressing a fusion protein containing an N-terminal human myelin associated glycoprotein (human MAG; NM_{—}002361; amino acids 1-516) and a C-terminal AP domain (amino acids 23-511), bearing six C-terminal histidine residues, was created (referred to as MAG-AP). Cells were incubated in serum-free medium for 48 hours, conditioned medium was collected, and the fusion protein was purified using TALON cobalt affinity chromatography (Clontech) following the manufacturer's protocol. MAG-AP concentration was determined by measuring alkaline phosphatase activity and by Western blot analysis for alkaline phosphatase and MAG.

### Example 1.8

### Immunoprecipitations and Western Blot Analysis

For initial studies, CHO-K1 cells (100 mm dishes) were transfected with p75NTR (see, e.g., Example 1.13), wild type NgR (see, e.g., Example 1.5), and various mutants of NgR1 (see, e.g., Example 1.5). The cells were harvested after 24 hours and lysed in 1 ml RIPA buffer (Sigma) supplemented with complete protease inhibitor cocktail (Roche Applied Science, Indianapolis, Ind.). After centrifugation at 14,000Xg for 15 minutes, the supernatants were collected and protein assay (Bio-Rad Laboratories, Hercules, Calif.) was performed. Protein lysates (0.5 mg) were preincubated with protein G-sepharose beads (GE Healthcare, Fairfield, Conn.) at 4° C. for 1 hour, then incubated with 2 μg of goat anti-human NgR1 antibody (R&D systems) plus protein G-sepharose at 4° C. overnight. The beads were washed three times with RIPA buffer and boiled in Laemmli sample buffer (Bio-Rad). Supernatants were subjected to 4-12% NuPAGE (Invitrogen), transferred onto nitrocellulose membranes (Bio-Rad) and probed with antibodies to NgR1 or p75NTR (Promega, Madison, Wis.). Western blot images were analyzed by the STORM™ gel and blot imaging system (GE Healthcare) and ImageQuant software (GE Healthcare).

For further studies, COS-7 cells transiently transfected with p75NTR, human wild type NgR1, or mutant NgR1 were lysed in SDS sample buffer and subjected to reducing SDS-gel electrophoresis on 4-12% LongLife gradient gels (Life Therapeutics, Clarkston, Ga.). Proteins were electrophoretically transferred to Hybond ECL membranes (Amersham Biosciences, Pittsburgh, Pa.) and blocked by incubation for one hour with Tris-buffered saline/0.1% Tween-20 (TBST) containing 5% dried milk powder (BLOTTO, Rockland Immunochemicals, Inc., Gilbertsville, Pa.). Membranes were then incubated in anti-NgR1 mouse monoclonal antibody (Reagent 645-1, Wyeth, Cambridge, Mass.) or anti-actin (1:5000) goat polyclonal antibody (Santa Cruz Biotechnology, Santa Cruz, Calif.) in BLOTTO for 1 hour at room temperature. Membranes were washed in Tris-buffered saline Tween-20 (TBST) and incubated with the appropriate peroxidase-conjugated, secondary antibody. Signals were developed using ECL Western Blotting Detection Reagents (Amersham) according to the manufacturer's instructions.

### Example 1.9

### Immunocytochemistry

COS-7 cells transiently transfected with human NgR1 were seeded into 8-well LAB-TEK™ CHAMBER SLIDE™ system glass slides (Nunc, Rochester, N.Y.). The next day, wells were rinsed three times with phosphate-buffered saline (PBS) and then fixed with 4% paraformaldehyde in PBS for twenty minutes at room temperature (RT). Following fixation, wells were rinsed three times with PBS and blocked with 3% donkey serum in PBS (blocking buffer) for 1 hour at RT. Following blocking, anti-NgR1 antibody (R&D Systems) diluted to a concentration of 100 ng/ml in blocking buffer was added to the wells and slides were incubated overnight at 4° C. The next morning, wells were washed three times for five minutes each with PBS followed by incubation for 40-60 minutes at RT with Cy3-conjugated, anti-goat IgG antibody (Jackson ImmunoResearch, West Grove, Pa.) diluted to a concentration of 5 μg/ml in PBS. Wells were then washed once with PBS containing 285 μM 4′,6-diamidino-2-phenylindole dihydrochloride (DAPI) at a final concentration of 285 μM for five minutes followed by three additional five minute washes with PBS. Slides were then disassembled, covered with Vecta-Shield mounting medium (Vector Labs, Burlingame, Calif.), coverslipped, and visualized using a Nikon Eclipse TE300 microscope equipped with an epi-fluorescent attachment, Spot-RT color digital camera, and Spot Advanced V4.0.5 software (Diagnostic Instruments, Sterling Heights, Mich.).

### Example 1.10

### Construction of NgR1(310)-fc Mutants

Human Nogo Receptor 1 (NgR1)(310)-fc point mutants (FRG-1 (R151E); FRG-2 (R279E); FRG-3 (R199E), and FRG-4 (R151E/R279E/R199E)) were constructed by Genewiz (North Brunswick, N.J.) using their site-directed mutagenesis technology. The template used to construct all the described mutants was wild type human NgR1(310)-fc (SEQ ID NO:59), which was generated by fusing a nucleotide sequence corresponding to the first 310 amino acid of Human Nogo Receptor 1 to a human Fc fragment in a mammalian expression vector (the sequence of wild type NgR1(310)-fc and the expression vector is set forth in SEQ ID NO:60 and a schematic of the expression vector is shown in FIG. 11). FRG-4 was a triple mutant that combined three single mutations, i.e., using all three sets of primers listed in Table 3 (below) to create the R151E, R279E, and R199E mutations. Mutagenic oligonucleotide sequences used were as follows in

TABLE 3

| | | SEQ |
|---|---|---|
| | | ID |
| Mutant | Primer sequence from 5′ to 3′ | NO: |
| FRG-1 | CTGGGCCCGGGGCTGTTCgagGGCCTGGCTGCCCTGCAG | 53 |
| | CTGCAGGGCAGCCAGGCCctcGAACAGCCCCGGGCCCAG | 54 |
| FRG-2 | GCCTGGCTGCAGAAGTTCgagGGCTCCTCCTCCGAGGTG | 55 |
| | CACCTCGGAGGAGGAGCCctcGAACTTCTGCAGCCAGGC | 56 |
| FRG-3 | GTGCCCGAGCGCGCCTTCgagGGGCTGCACAGCCTCGAC | 57 |
| | GTCGAGGCTGTGCAGCCCctcGAAGGCGCGCTCGGGCAC | 58 |

### Example 1.11

### Analytical Ultracentrifugation

Sedimentation velocity experiments were performed on a Beckman XLI/XLA analytical ultracentrifuge. Wild type NgR(310)-fc (0.21 μM to 0.38 μM final) was added to ganglioside at concentrations increasing from 0 to 48 μM. Mutant protein used in the sedimentation velocity experiments corresponded to the column fraction of greatest purity based on SDS gel analysis. Wild type or mutant NgR(310)-fc was added to TBS buffer or TBS buffer containing GT1b to a final concentration of 16 to 30 μg/mL protein and or 22 μM GT1b in a microfuge tube. The solution (400 μL) was loaded into two-channel (1.2 cm path length) carbon-Epon centerpieces in an An-50-Ti rotor. Scans were recorded at 20° C. with a rotor speed of 35,000 rpm, and the signal was detected at 230 nm with a spacing of 0.006 in the continuous mode. Sedimentation profiles were analyzed by the program Sedfit (Schuck (2000) Biophys. J. 78:1606-19) to obtain the sedimentation coefficient distributions. The solvent density (1.006) and partial specific volume (0.72) were calculated using the program Sednterp (Laue et al. (1992) Analytical Ultracentrifugation in Biochemistry and Polymer Science (Harding, S. E., Towe, A. J. and Horton, J. C., eds.) pp. 90-125, Royal Society of Chemistry, Cambridge, U.K).

### Example 1.12

### PharmDock Screening

The protein structure of the NgR1 ligand-binding domain was examined to identify potential small-molecule binding sites or pockets on the surface. A number of computational approaches were employed to visualize and analyze the structure (e.g., the GRID program (Goodford (1985) J. Med. Chem. 28:849-57, the MCSS program (Miranker and Karplus (1991) Proteins 11:29-34; Evensen et al. MCSSv2; 2.1 ed.; Harvard University: Cambridge, Mass.), and the MOE site finder method (Chemical Computing Group, Montreal, Quebec, Canada, 2005)). Virtual screens focusing on the most promising binding sites were carried out to identify small molecule ligands capable of binding to NgR1 and blocking or attenuating the interaction of NgR1 with its natural protein ligands. More specifically, PharmDock (Joseph-McCarthy et al. (2003) Proteins 51:189-202; Joseph-McCarthy et al. (2003) Proteins 51:172-88) was used to search large molecular databases for potential binders to NgR1.

### Example 1.13

### Cell-surface p75NTR-NgR-AP Binding Assay

NgR-AP was collected from CHO-K1 cells expressing NgR-AP (CHO-NgR-AP). Briefly, the growth medium of CHO-NgR-AP cells grown to 90-95% confluence in T175 flasks was replaced with 25 ml of serum-free medium, R5CD1. After 48 hours, the medium was collected and concentrated 4× using an Amicon Ultra filtration device. Fifty μl of concentrated NgR-AP was added to each well of a 96-well black/clear bottom plate that had been seeded with 30,000-CHO-K1 cells expressing cell-surface human p75NTR (CHO-p75NTR) the day before. The plates were incubated on a shaker at RT for 90 minutes, and each well was gently washed four times with 250 μl HBSH (HBSS, 1% serum, 20 mM HEPES). AltoPhos (100 μl) at a concentration of 0.6 mg/ml was added and color was developed for 30 minutes before plates were endpoint read with FLEXSTATION® (Molecular Devices, Sunnyvale, Calif.) at an excitation wavelength of 435 nm and emission wavelength of 555 nm. About five-fold more NgR-AP was detected bound toCHO-p75NTR cells compared to control mock-transfected CHO-K1 cells not expressing p75NTR on the cell surface (data not shown).

### Example 1.14

### Neuraminidase Treatment

Chinese hamster ovary (CHO) parental cells and NgR1 stable cells were seeded at 30,000 cells per well in 96-well plates the night before the assay. Various concentrations of Vibrio cholera neuraminidase (Roche Applied Science, Indianapolis, Ind.) in growth medium (Dulbecco's modified Eagle medium containing 10% fetal bovine serum) were incubated with cells for an hour at 37° C. Medium was replaced with affinity-purified MAG-AP or Nogo66-AP in HBSS supplemented with 1% fetal bovine serum and 20 nM of HEPES and incubated at room temperature for 90 min. Cells were then washed four times with supplemented HBSS. AltoPhos (0.6 mg/ml) (Promega, Madison Wis.) was added for indication of bound ligands. After a 30 minute incubation at RT, the plates were read at emission/excitation wavelength of 400 nm/505 nm with FLEXSTATION® 11384.

### Example 1.15

### Testing Identified Compounds

The cell-surface NgR binding and/or the cell-surface p75NTR-NgR-AP binding assays are used to test potential antagonists (e.g., pharmacological agents or lead compounds) to NgR1 ligands (e.g., myelin-associated glycoprotein, oligodendrocyte myelin glycoprotein, Nogo-A, Nogo-66, GT1b, an antibody to Nogo receptor, an antibody to GT1b, an antibody to p75 neurotrophin receptor, and an antibody to Lingo-1) which may be used to antagonize (e.g., reverse, decrease, reduce, prevent, etc.) NgR1-mediated inhibition of axonal growth. For example, samples containing cells expressing NgR or p75NTR on the cell surface (as disclosed herein) and an NgR1 ligand (including MAG-AP, Nogo-AP) or a p75NTR ligand (e.g., NgR-AP), respectively, are contacted with one of a plurality of test compounds, and the interaction of cell-surface NgR1 or p75NTR to the respective NgR1 or p75NTR ligand can be compared to the interaction of cell-surface NgR1 or p75NTR to the respective NgR1 or p75NTR ligand in untreated samples or in samples contacted with different test compounds to determine whether any of the test compounds provides a substantially decreased level of NgR1:NgR1 ligand or p75NTR:p75NTR ligand interactions. A potential antagonist capable of decreasing levels of NgR1:NgR1 ligand or p75NTR:p75NTR ligand interactions is further tested for its ability to antagonize NgR1L-mediated axonal growth inhibition using, e.g., the neurite outgrowth assay described in Example 1.1. Upon confirmation that the tested agent or compound is an antagonist, the compound is used in methods of treating, ameliorating, preventing, diagnosing, prognosing, or monitoring disorders arising from inhibition of axonal growth mediated by the binding of NgR1 ligands to NgR1.

### Example 2

### Results

### Example 2.1

### Binding Motifs on the NgR1

There are two published crystal structures of NgR1, protein data bank accessions 10ZN (He et al. (2007) supra) and 1P8T (Barton et al. (2003) supra) but currently no ligand-receptor complex structure has been solved. However, with the knowledge of the receptor structure, the protein ligand binding site can be predicted using a potential of mean force calculation (Williams (2006) Online J. Bioinformatics 7:32-34). The potential is determined by the distribution of relative separations and angular orientations of pairs of residue centroids within a representative set of crystal structures. Local energy minima experienced by a general residue probe as it moves over the surface of the receptor are calculated, and it is predicted that the dominant cluster of minima corresponds to the protein ligand binding site. The predicted NgR1 protein ligand binding site is shown in FIG. 1A. Detailed mutagenesis studies have recently mapped the residues critical for the binding of all three myelin inhibitors (Lauren et al (2007) J. Biol. Chem. 282:5715-25), and these correspond with a high degree of accuracy to the predicted protein-protein interaction face (FIG. 1A).

Small ligand binding sites show up as cavities and can be revealed by the clustering of a small probe under the influence of a van der Waals potential. In FIG. 1B, the two lowest energy clusters for a probe with van der Waals radius of 3.5 Å are shown. The potential binding pockets lie on the convex side of the protein and, interestingly, both pockets neighbor FRG triplet motifs that can be found in the other NgRs (discussed further herein). These data suggest that the NgR has the capacity to bind small ligands at sites neighbored by conserved FRG motifs. One possibility is that these are sites for ganglioside interactions with the NgR; this is supported by the fact that sialic acid binds to an FGR motif in MAG itself (Tang et al. (1997) Mol. Cell. Neurosci. 9:333-46). Thus, the inventors speculated that the equivalent loops on the NgR1 might be important for ligand binding and/or the formation of a higher-order signaling complex. Although the NgR1 has one extra LRR motif relative to glycoprotein Ib alpha, the two structures are quite similar (data not shown). In glycoprotein Ib alpha, the N- and C-terminal exposed loops are crucial to the interaction with the ligand. Based on this analysis, the equivalent loops and a number of putative functional motifs on the NgR1 were hypothesized, as shown in FIG. 1. These are exposed sites that, based on homology, might be expected to engage in protein-protein interactions. Peptide mimetics of binding motifs in proteins often function as antagonists in biological assays, particularly if they are constrained by a disulfide bond (see, e.g., Williams et al. (2000) supra; Williams et al. (2000a) supra). Thus, cyclic peptide mimetics of the four putative and/or actual motifs on the NgR1 that are highlighted in FIG. 1C were designed. These peptides were coded as follows:

NRL1 (N-Ac-CYNEPKVTC-NH₂ (SEQ ID NO:27)),

NRL2 (N-Ac-CLQKFRGSSC-NH₂ (SEQ ID NO:31)),

NRL3 (N-Ac-CSLPQRLAC-NH₂ (SEQ ID NO:28)) and

NRL4 (N-Ac-CAGRDLKRC-NH₂ (SEQ ID NO:30)).

### Example 2.2

### Binding of MAG, But Not Nogo66, to NgR1 is Partially Sensitive to Neuraminidase

In neurons, soluble MAG binds to the NgR1 and NgR2 in a sialic acid-dependent manner (Venkatesh et al. (2005) supra). In the present study, the neuraminidase sensitivity of MAG binding to the NgR1 expressed in CHO cells was confirmed. The data show that over a wide range of concentrations (2.5-20 μg/ml) the specific binding of the MAG-AP fusion protein to NgR1-expressing cells is partially inhibited (55%) by treating the CHO cells with neuraminidase. The effect was dependent upon the concentration of neuraminidase, and even at the highest concentration, Nogo66-AP remained completely unaffected (FIG. 2). These data suggest that MAG binding to the NgR1 is only partially dependent on sialic acid binding.

### Example 2.3

### Effects of Loop 2 and Additional FRG Mutations on GT1b Binding to the NgR1

The peptide competition studies, together with the direct binding assays, have implicated the FRG motif within loop 2 as being important for MAG function. MAG also contains an FRG motif that forms part of a sialic acid binding site that can recognize a variety of ligands, including GT1b (Vinson et al. (2001) supra; Tang et al. (1997a) supra). GT1b is sialic acid-containing ganglioside that has previously been reported to be a key component of the MAG receptor (Vyas et al. (2002) supra; Yamashita et al. (2002) supra), and on this basis the inventors speculated that the NgR1 might also use FRG motifs to bind GT1b. Importantly, there are three FRG motifs in the NgR1. In the present study, analytical ultracentrifugation was performed to determine whether GT1b can bind directly to the ectodomain of the NgR1. In the absence of GT1b, the dimeric NgR1(310)-fc migrates with a sedimentation coefficient of ca. 6.5 S (FIG. 3). In the presence of low μM concentrations of GT1b, the 6.5 S species decreases and additional peaks with higher sedimentation coefficients appear in a dose-dependent manner (FIG. 3A). In this assay, GM1 can also interact with NgR1 (FIG. 3B) and this implicates the common terminal α2,3-linked sialic acid shared by GT1b GM1 in the interaction. GT1b and GM1 form micelles at the concentrations used in this study (Formisano et al. (1979) Biochemistry 18(6): 1119-24) that migrate with a sedimentation coefficient of ca. 4.5 corresponding to approximately 10-12 molecules per micelle. No change in sedimentation coefficient of NgR1(310)-fc is observed in the presence of asialo-GM1, indicating that the binding is specific to sialic acid-containing gangliosides and not solely due to nonspecific binding of NgR1 to the ganglioside micelle (FIG. 3C). No change in sedimentation coefficient of the NgR1(310)-fc is observed in the presence of asialo-GM1. No effect was observed upon addition of 22 mM GT1b to anti-hNgR AF 1208 antibody (R&D) and this provides additional evidence that the interaction of GT1b with NgR1 is specific (data not shown).

Further experiments determined whether the binding of GT1b to the NgR1 was sensitive to mutation of the FRG motifs. Importantly, based on the relative ratios of the ˜6.7 S and ˜11 S peaks, it can be estimated that mutation of the arginine 279 to an aspartic acid reduced binding to approximately 56% of wild type NgR, suggesting this site plays a role in mediating the interaction (FIG. 3D). Mutation of arginine 151 (FIG. 3E) or arginine 199 (FIG. 3F) also reduced GT1b binding to 49% and 33% of wild type, respectively. These data suggest that all three FRG sites might be important in facilitating GT1b binding to NgR1. In all three instances, the sedimentation coefficient curves can be seen to be qualitatively different for the curve seen with the wild type NgR1 construct. Whereas a higher migrating species (˜11 S) becomes the dominant species in the presence of GT1b with the wild type receptor, lower migrating species remain dominant with all three mutated receptors (FIGS. 3D-F).

### Example 2.4

### An FRG-containing Mimetic of an NgR1 Loop Inhibits the Function of a GT1b Antibody

In general, antibodies that bind to cerebellar neurons do not inhibit neurite outgrowth (including antibodies to NCAM, N-cadherin, L1 and the FGFR (see, e.g., Williams et al. (1994) supra). However, antibodies that cluster GT1b inhibit neurite outgrowth, most likely by clustering GT1b with consequent clustering and activation of the NgR complex (Vyas et al. (2002) supra; Fujitani et al. (2005) supra; Vinson et al. (2001) supra; Williams et al. (2005) supra). One of the FRG motifs implicated in GT1b binding to the NgR1 is part of an exposed loop that lends itself well to the design of a cyclic peptide mimetic (see FIG. 1C). In the present study, post-natal day (PND) ⅔ cerebellar neurons were cultured over monolayers of 3T3 fibroblasts for ˜23 hrs in the presence and absence of a GT1b antibody. As previously reported, the antibody inhibits neurite outgrowth in a dose-dependent manner with a robust inhibition seen at 40 μg/ml (FIGS. 4A and 4B). When the antibody was added in the presence of 100 μg/ml of a cyclic peptide (N-Ac-CLQKFRGSSC-NH2) that mimicked the FRG motif-containing loop (the NRL2 peptide), it failed to inhibit neurite outgrowth as tested at up to 40 μg/ml (FIG. 4B). Showing that an NgR1-derived peptide can inhibit the GT1b antibody response further substantiates the hypothesis that the GT1b antibody response might rely on GT1b binding to the FRG motifs in the NgR.

### Example 2.5

### Effects of the NRL2 Peptide on MAG Inhibition of Neurite Outgrowth

A wide range of Fc-chimeras that bind to neurons do not inhibit neurite outgrowth (Williams et al. (1994) supra; Meiri et al. (1998) J. Neurosci. 18:10429-37; Doherty et al. (1998) Neuron 14:57-66). In contrast, a soluble MAG-Fc chimera inhibits neurite outgrowth in a manner that depends upon both ganglioside and NgR function. In the present study, the MAG-Fc inhibited neurite outgrowth from PND ⅔ cerebellar neurons in a dose-dependent manner (data not shown) with a robust inhibition seen at 25 μg/ml (FIG. 5A). The NRL2 peptide again had no effect on basal neurite outgrowth, but it was striking that the MAG-Fc failed to substantially inhibit neurite outgrowth when this peptide was present in the growth media (FIG. 5A). As a control, we tested cyclic versions of the three other exposed NgR loops (see Example 2.1 for details) for their effects on neurite outgrowth. These peptides were coded NRL1 (N-Ac-CYNEPKVTC-NH2), NRL3 (N-Ac-CSLPQRLAC-NH2), and NRL4 (N-Ac-CAGRDLKRC-NH2). When tested at 100 μg/ml, these peptides had no effect on basal neurite outgrowth, or on the suppressed neurite outgrowth seen in the presence of MAG-Fc. Next, the dose-response curve for the NRL2 peptide was examined; no significant effect on neurite outgrowth in control media was seen when tested at up to 200 μg/ml. In contrast, the peptide promotes neurite outgrowth in a dose-dependent manner in the presence of the MAG Fc, with the response reaching a plateau at around 50 μg/ml (˜45 mM) (FIG. 5B).

### Example 2.6

### Identification of Key Functional Amino Acids in the NRL2 Sequence

Structural analyses of the NgR1 show that the most conspicuous amino acids within the loop corresponding to the NRL2 peptide sequence are the positively charged lysine (K) and arginine (R); both are highly solvent exposed, with their side chains clearly available for binding (data not shown). Of the surrounding amino acids, the phenylalanine (F) is buried in the structure, but might play a role in stabilizing the local region. The glycine and serine are partially solvent exposed, but look less likely as candidates to mediate a binding interaction. Based on this analysis, two small peptides that both have the key lysine and arginine within them were designed. These were NRL2a (N-Ac-CKFRGSC-NH₂ (SEQ ID NO:32)) and NRL2b (N-Ac-CQKFRGC-NH₂ (SEQ ID NO:33)) peptides; both peptides contain a common four amino acid motif (KFRG (SEQ ID NO:26)). Both peptides had no effect on neurite outgrowth in control (i.e., without MAG-Fc) media (data not shown); their ability to antagonize NgR1-ligand-mediated inhibition of axonal growth, i.e., to “promote” growth in the presence of the MAG-Fc, is shown in FIG. 6A. Within the inhibitory environment, both peptides “promoted” neurite outgrowth, with significant effects seen at 25 μg/ml (30 μM) and maximal effects seen at 50 μg/ml (60 μM). At this higher concentration, the inhibitory activity of the MAG-Fc was effectively antagonized (i.e., decreased, reduced, abolished, prevented, etc.). This suggests that the functional activity within the NRL2 peptide sequence resides within the KFRG motif.

In order to identify key amino acids within this short region, four peptides (N-Ac-CQAFRGC-NH₂ (SEQ ID NO:46); N-Ac-CQKARGC-NH₂ (SEQ ID NO:47); N-Ac-CQKFAGC-NH₂ (SEQ ID NO:48); N-Ac-CQKFRAC-NH₂ (SEQ ID NO:49)) with individual alanine substitutions within the KFRG sequence of the NRL2b peptide were synthesized and tested for their ability to antagonize MAG-Fc-mediated inhibition of axonal growth. When tested at 100 μg/ml, peptides with alanine substitutions at position 1 (N-Ac-CQAFRGC-NH₂ (SEQ ID NO:46) or position 2 (N-Ac-CQKARGC-NH₂ (SEQ ID NO:47)) were as effective as NRL2b in antagonizing MAG-mediated inhibition of axonal growth (FIG. 6B). When tested over a range of concentrations, substitution at position 1 had no obvious effect on the efficacy of the peptide (FIG. 6C), whereas substitution at position 2 reduced efficacy by about two-fold at 25-50 μg/ml (FIG. 6D). In contrast, alanine substitutions at position 3 (N-Ac-CQKFAGC-NH₂ (SEQ ID NO:48)) or position 4 (N-Ac-CQKFRAC-NH₂ (SEQ ID NO:49)) rendered the peptides ineffective at antagonizing MAG-mediated inhibition of axonal growth (FIG. 6B). Also, a linear version of the QKFRG (SEQ ID NO:22) peptide did not antagonize MAG-mediated inhibition of axonal growth (FIG. 6B). These data demonstrate that in order to be functional, the QKFRG motif needs to be constrained by a disulfide bond, and that single mutations to any amino acid within the FRG motif compromises activity of the peptide.

In order to determine if a relatively metabolically stable peptide would retain biological activity, the NgR1 sequence was cyclized via a stable peptide bond (homodetic cyclization), and the amino acids were replaced by their chiral partners. Specifically, the L-type amino acids of the original peptide were replaced by normative D-type amino acids. The peptide sequence was reversed to ensure that the side-chain orientations were preserved. Such peptides are referred to as retro-inverso peptides. Explicitly, the sequence of the homodetic retro-inverso peptide (hriNRL2) is c[sGrfkq], where c[ ] refers to homodetic cyclization and the lower case letters refer to D-type amino acids (note that glycine has no chirality as it has no side chain). When tested in the MAG-Fc assay, this peptide can be seen to retain full efficacy in inhibiting the MAG response (FIG. 6E).

Neuraminidase inhibits the function of soluble, but not substrate-bound MAG (Tang et al. (1997a) supra; DeBellard et al. (1996) Mol. Cell. Neurosci. 7:89-101). This was interpreted as suggesting that soluble MAG requires a sialic acid-containing coreceptor for maximal efficacy. Interestingly, the function of substrate-bound MAG was not inhibited with any of the NRL2 peptides; this is shown for the hriNRL2 peptide in FIG. 6F. In this example, the hriNRL2 peptide had no significant effect on neurite outgrowth when tested at up to 200 μg/ml on the suppressed growth that is seen on the MAG substrate. The NRL2 peptides do not promote growth over substrate-bound myelin (data not shown), confirming that they do not have nonspecific effects on neurite outgrowth.

### Example 2.7

### Effects of Loop 2 Mutations on Ligand Binding to the NgR1

The data suggest that the 277KFRK280 motif in loop 2 in the NgR1 plays an important role in the context of soluble, but not substrate-bound, MAG function. Given that the lysine 277 and arginine 279 are positively charged and highly solvent-exposed, the effects of mutating both residues to negatively charged aspartic acids, or neutral alanines, was determined. In both instances, the mutations had no obvious effect on the level of expression of the NgR1 (FIG. 7A), and based on coimmunoprecipitation, a normal interaction between the mutated NgR1 constructs and the p75NTR, presumably in the cell membrane, was apparent. The p75NTR did not coimmunoprecipitate with a control antibody (data not shown). When soluble MAG was tested in binding assays, a significant reduction in binding (˜60%) was seen to the mutated NgRs (EM7=277D/R279D, 57%, p<0.008; EM8=227A/R279A, 58%, p<0.002) irrespective of whether the exposed lysine and arginine were substituted with aspartic acids or alanines (FIG. 7B). When these positively charged amino acids were individually mutated to alanines, the data suggested that arginine 279 is more important for MAG-AP binding than lysine 277 (FIG. 7B) with a 36% reduction (p<0.02) in binding seen following this former single-point mutation. The same mutations had little or no significant effects on the ability of the NgR1 constructs to bind Nogo-66-AP (FIG. 7B) or p75NTR (FIG. 7C).

### Example 2.8

### Modeling and Virtual Screening of NgR1 for Compound Antagonists

The surface features on NgR1 present virtual screening opportunities; for example, the side surface of NgR1 was shaded by hydrophobicity and presented a putative binding pocket based on the size and depth of cavity (FIG. 8). Additionally, there was a convergence between the functionally validated NRL2 peptide site and putative binding pocket on the side surface of NgR1 (FIG. 9), indicating that the side-binding pocket and/or NRL2 are functional motifs. The identification of this pocket and/or the favored binding region within this site permitted a strategy for screening compounds capable of antagonizing NgR1 ligand-mediated inhibition of axonal growth in a sample or subject, e.g., a PharmDock query on the side-binding pocket. A lead-like corporate database was docked inside grid-based fields within a box defined around the binding pocket/functional motif. Compounds that matched favored binding regions were selected and scored based on chemical forces within the site. Examples of such compounds are shown in FIG. 10.

What is claimed is:

1. An antagonist to an NgR1 ligand comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

2. The antagonist as in claim 1, wherein the antagonist comprises at least one D-amino acid.

3. The antagonist of claim 2, wherein the polypeptide comprises the amino acid sequence of SEQ ID NO:37 or an active fragment(s) thereof.

4. A method of screening for compounds that compete with antagonists of NgR1 ligands comprising the steps of:
(a) contacting a sample containing an NgR1 ligand and an antagonist with a compound, wherein the antagonist comprises a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof; and
(b) determining whether the interaction between the NgR1 ligand and the antagonist in the sample is decreased relative to the interaction of the NgR1 ligand and the antagonist in a sample not contacted with the compound,
wherein a decrease in the interaction of the NgR1 ligand and the antagonist in the sample contacted with the compound identifies the compound as one that competes with the antagonist.

5. The method of claim 4, wherein the compound is further identified as one that antagonizes at least one NgR1 ligand.

6. A method of antagonizing inhibition of axonal growth in a sample comprising the step of contacting the sample with an antagonist to at least one NgR1 ligand.

7. A method of antagonizing inhibition of axonal growth in a sample comprising the step of contacting the sample with an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

8. A method of antagonizing inhibition of axonal growth in a subject comprising the step of administering to the subject an effective amount of an antagonist to at least one NgR1 ligand.

9. A method of antagonizing inhibition of axonal growth in a subject comprising the step of administering to the subject an effective amount of an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

10. The method of claim 9, wherein the inhibition of axonal growth is mediated by at least one NgR1 ligand.

11. The method of claim 9, wherein the antagonizing of inhibition of axonal growth results in regeneration of axons.

12. A pharmaceutical composition comprising a pharmaceutically acceptable carrier and an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

13. An antagonist to an NgR1 ligand comprising a polypeptide comprising an amino acid sequence selected from the group consisting of the amino acid sequence of SEQ ID NO:2, the amino acid sequence of SEQ ID NO:4, the amino acid sequence of SEQ ID NO:6, the amino acid sequence of SEQ ID NO:10, and the amino acid sequences of active fragments thereof.

14. An isolated antibody capable of specifically binding to a polypeptide comprising an amino acid sequence selected from the group consisting of the amino acid sequences of SEQ ID NOs:2, 4, 6, 8, 10, 12, 14, 16, 18, 20, 22, 24, 26, 27, 28, 29, 30, 31, 32, 33, 34, 37, and the amino acid sequences of active fragments thereof.

15. An isolated antibody capable of specifically binding to an antagonist to at least one NgR1 ligand.

16. An NgR1 functional motif comprising the amino acid sequence FRG.

17. An antagonist to the NgR1 functional motif of claim 16.

18. The antagonist of claim 17, wherein the antagonist is selected from the group consisting of WAY-100080, WY-48185, WY-23626, CL-391991, CL-306115, and WY-46543.

19. A method of determining whether a compound inhibits an NgR1 ligand from binding NgR1 comprising the steps of:
(a) contacting a sample containing an NgR1 ligand and NgR1 with a compound; and
(b) determining whether the interaction between the NgR1 ligand and NgR1 is decreased relative to the interaction of the NgR1 ligand and NgR1 in a sample not contacted with the compound,
wherein a decrease in the interaction of the NgR1 ligand and NgR1 in the sample contacted with the compound identifies the compound as one that inhibits an NgR1 ligand from binding NgR1.

20. A method of treating a subject with a neurodegenerative disorder comprising the step of antagonizing NgR1.

21. The method of claim 20 wherein the step of antagonizing NgR1 comprises inhibiting an NgR1 ligand from binding NgR1.

22. The method of claim 20, wherein the step of antagonizing NgR1 comprises administering to the subject an antagonist of NgR1.
